(19) United States
(12) Patent Application Publication
(10) Pub. No.: US 2026/0198620 A1
(43) Pub. Date: Jul. 16, 2026

(54) Smart Bra with Optical Sensors and Expandable Chambers for Detecting Abnormal Breast Tissue

(71) Applicant: Robert A. Connor, Wyoming, MN (US)

(72) Inventor: Robert A. Connor, Wyoming, MN (US)

(73) Assignee: Holovisions LLC, Ham Lake, MN (US)

(21) Appl. No.: 19/577,512

(22) Filed: Mar. 25, 2026

Related U.S. Application Data
(63) Continuation-in-part of application No. 18/964,893, filed on Dec. 2, 2024.
(63) Continuation-in-part of application No. 18/666,352, filed on May 16, 2024, now abandoned.
Application No. 18/964,893 is a continuation-in-part of application No. 18/666,352, filed on May 16, 2024, now abandoned.
Application No. 18/666,352 is a continuation-in-part of application No. 18/237,231, filed on Aug. 23, 2023, now abandoned.
Application No. 18/237,231 is a continuation-in-part of application No. 18/096,748, filed on Jan. 13, 2023, now Pat. No. 12,201,402.
Application No. 18/096,748 is a continuation-in-part of application No. 18/237,231, filed on Aug. 23, 2023, now abandoned.
Application No. 18/237,231 is a continuation-in-part of application No. 18/096,748, filed on Jan. 13, 2023, now Pat. No. 12,201,402.
Application No. 18/096,748 is a continuation-in-part of application No. 17/897,182, filed on Aug. 28, 2022, now Pat. No. 11,950,881.
Application No. 17/897,182 is a continuation-in-part of application No. 16/933,138, filed on Jul. 20, 2020, now abandoned.
(60) Provisional application No. 62/879,485, filed on Jul. 28, 2019.
(60) Provisional application No. 63/648,156, filed on May 15, 2024.
(60) Provisional application No. 63/648,156, filed on May 15, 2024.

(51) Int. Cl. A41C 3/00; A41C 3/14; A61B 5/00

(52) U.S. Cl. CPC A41C 3/0064; A41C 3/14; A61B 5/0091; A61B 5/6823; A61B 2560/0462; A61B 2562/0238

(57) ABSTRACT

A smart bra with optical sensors for detecting, imaging, and/or evaluating abnormal breast

tissue includes a cup with a proximal outer ring, expandable chambers inside the proximal outer ring, and light emitters and light receivers between the expandable chambers and a person's breast. Changes in light emitted from the light emitters and received by the light receivers which are caused by transmission of the light through breast tissue can be analyzed to detect, image, and evaluate abnormal breast tissue.

### CROSS-REFERENCE TO RELATED APPLICATIONS

This application is a continuation-in-part of U.S. application Ser. No. 18/964,893 filed on 2024 Dec. 02. This application is a continuation-in-part of U.S. application Ser. No. 18/666,352 filed on 2024 May 16. U.S. application Ser. No. 18/964,893 was a continuation-in-part of U.S. application Ser. No. 18/666,352 filed on 2024 May 16. U.S. application 18/964,893 claimed the priority benefit of U.S. provisional application 63/648,156 filed on 2024 May 15. U.S. application Ser. No. 18/964,893 was a continuation-in-part of U.S. application Ser. No. 18/237,231 filed on 2023 Aug. 23. U.S. application Ser. No. 18/964,893 was a continuation-in-part of U.S. application Ser. No. 18/096,748 filed on 2023 Jan. 13.

U.S. application Ser. No. 18/666,352 claimed the priority benefit of U.S. provisional application 63/648,156 filed on 2024 May 15. U.S. application Ser. No. 18/666,352 was a continuation-in-part of U.S. application Ser. No. 18/237,231 filed on 2023 Aug. 23. U.S. application Ser. No. 18/237,231 was a continuation-in-part of U.S. application Ser. No. 18/096,748 filed on 2023 Jan. 13. U.S. application Ser. No. 18/096,748 was a continuation-in-part of U.S. application Ser. No. 17/897,182 filed on 2022 Aug. 28 which issued as U.S. Pat. No. 11,950,881 on 2024 Apr. 09. U.S. application Ser. No. 17/897,182 was a continuation-in-part of U.S. application Ser. No. 16/933,138 filed on 2020 Jul. 20. U.S. application Ser. No. 16/933,138 claimed the priority benefit of U.S. provisional application 62/879,485 filed on 2019 Jul. 28. The entire contents of these related applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to wearable devices for medical imaging and diagnosis.

### INTRODUCTION

Early detection of breast cancer is vital. Current methods for breast cancer detection include x-ray mammography, MRI, and ultrasound. These methods have limitations. X-ray mammography causes exposure to ionizing radiation and is not very mobile. MRI equipment is expensive and not very mobile. Ultrasound can be labor-intensive and have low sensitivity. Optical scanning with near-infrared light and spectroscopic analysis is another method for breast cancer detection which can address these limitations. Although unlikely to replace traditional modalities such as x-ray mammography in geographic regions with good access to large radiological equipment, optical scanning may provide women with a useful supplemental technology for earlier and/or more-frequent screening with less exposure to ionizing radiation and less discomfort.

Several biomarkers are present in different concentrations in abnormal tissue. These include deoxygenated hemoglobin, oxygenated hemoglobin, lipids, collagen, oxygen, and water. Changes in light transmission through breast tissue caused by these biomarkers can be used to detect, image, and evaluate abnormal breast tissue. A smart bra with optical sensors for identification of abnormal breast tissue can help to ensure consistent optical sensor placement on a person's body over time (e.g. in periodic screenings) for enhanced longitudinal tracking and can enable gentle breast compression for scanning which is controlled by the person wearing the bra.

### REVIEW OF THE RELEVANT ART

U.S. patent application 20050043596 (Chance, Feb. 24, 2005, “Optical Examination Device, System and Method”) discloses a brush-form optical coupler with freely extending fiber end portions, sized and positioned to make optical contact with a subject, examination, and monitoring systems utilizing one or more of such couplers. U.S. patent application 20060058683 (Chance, Mar. 16, 2006, “Optical Examination of Biological Tissue Using Non-Contact Irradiation and Detection”) and U.S. Pat. No. 7,904,139 (Chance, Mar. 8, 2011, “Optical Examination of Biological Tissue Using Non-Contact Irradiation and Detection”) disclose an optical system for examination of biological tissue which includes a light source, a light detector, optics and electronics.

U.S. Pat. No. 6,081,322 (Barbour, Jun. 27, 2000, “NIR Clinical Opti-Scan System”) and RE38800 (Barbour, Sep. 20, 2005, “NIR Clinical Opti-Scan System”) disclose three-dimensional optical imaging techniques for the detection and three-dimensional imaging of absorbing and/or scattering structures in complex random media, such as human body tissue, by detecting scattered light. U.S. patent application 20150182121 (Barbour, Jul. 2, 2015, “Low-Cost Screening System for Breast Cancer Detection”) discloses a portable and wearable tumor detector including a brassier and devices for optical tomography. U.S. patent application publication 20150119665 (Barbour et al., Apr. 30, 2015, “Self-Referencing Optical Measurement for Breast Cancer Detection”) and U.S. Pat. No. 9,724,489 (Barbour et al., Aug. 8, 2017, “Self-Referencing Optical Measurement for Breast Cancer Detection”) disclose obtaining optical data from a pair of breasts, employing a simultaneous bilateral referencing protocol, and employing a self-referencing data analysis method.

U.S. patent applications 20100292569 (Hielscher et al., Nov. 18, 2010, “Systems and Methods for Dynamic Imaging of Tissue Using Digital Optical Tomography”) and 20150223697 (Hielscher et al., Aug. 13, 2015, “Systems and Methods for Dynamic Imaging of Tissue Using Digital Optical Tomography”) disclose methods for imaging tissue using diffuse optical tomography including directing a amplitude modulated optical signals from optical signal sources. U.S. patent application 20140330116 (Hielscher et al., Nov. 6, 2014, “Systems and Methods for Simultaneous Multi-Directional Imaging for Capturing Tomographic Data”) discloses devices, systems, and method for tomographic imaging in which light transmitted and backscattered surface light is imaged by an optical system that minimizes reflection back to the target object. U.S. patent applications 20130289394 (Hielscher et al., Oct. 31, 2013, “Dynamic Optical Tomographic Imaging Devices Methods and Systems”), 20170027480 (Hielscher et al., Feb. 2, 2017, “Dynamic Optical Tomographic Imaging Devices Methods and Systems”), and 20190282134 (Hielscher et al., Sep. 19, 2019, “Dynamic Optical Tomographic Imaging Devices Methods and Systems”), and U.S. Pat. No. 10,178,967 (Hielscher et al., Jan. 15, 2019, “Dynamic Optical Tomographic Imaging Devices Methods and Systems”) disclose an optical tomographic systems for acquiring and displaying dynamic data representing changes in a target tissue sample to external provocation. U.S. patent applications 20130338496 (Hielscher et al., Dec. 19, 2013, “Medical Imaging Devices, Methods, and Systems”) and 20140088415 (Hielscher et al., Mar. 27, 2014, “Medical Imaging Devices, Methods, and Systems”) disclose devices, methods, and systems for generating optical tomographic data including volumetric and surface geometric data.

U.S. patent application publication 20140236003 (Hielscher et al., Aug. 21, 2014, “Interfacing Systems, Devices, and Methods for Optical Imaging”) discloses an imaging interface with a plurality of concentric rings for diffuse optical tomography of breast tissue. U.S. patent applications 20140243681 (Hielscher et al., Aug. 28, 2014, “Compact Optical Imaging Devices, Systems, and Methods”) and 20190239751 (Hielscher et al., Aug. 8, 2019, “Compact Optical Imaging Devices, Systems, and Methods”), and U.S. Pat. No. 10,111,594 (Hielscher et al., Oct. 30, 2018, “Compact Optical Imaging Devices, Systems, and Methods”) disclose a handheld optical imaging system with a plurality of detectors. U.S. patent application 20150286785 (Hielscher et al., Oct. 8, 2015, “Systems, Methods, and Devices for Image Reconstruction Using Combined PDE-Constrained and Simplified Spherical Harmonics Algorithm”) and U.S. Pat. No. 9,495,516 (Hielscher et al. , 11/15/2016, “Systems, Methods, and Devices for Image Reconstruction Using Combined PDE-Constrained and Simplified Spherical Harmonics Algorithm”) disclose systems, methods, and devices for image reconstruction using combined PDE-constrained and simplified spherical harmonics (SPN) algorithms. U.S. Pat. No. 10,376,150 (Hielscher et al., Aug. 13, 2019, “Interfacing Systems, Devices, and Methods for Optical Imaging”) discloses an imaging interface for diffuse optical tomography of breast with a plurality of concentric rings.

U.S. patent application publication 20140236021 (Islam, Aug. 21, 2014, “Near-Infrared Super-Continuum Lasers for Early Detection of Breast and Other Cancers”) and U.S. Pat. No. 9,993,159 (Islam, Jun. 12, 2018, “Near-Infrared Super-Continuum Lasers for Early Detection of Breast and Other Cancers”) disclose a system and method using near-infrared or short-wave infrared light sources for early detection and monitoring of breast cancer. U.S. patent application publication 20180289264 (Islam, Oct. 11, 2018, “High Signal-to-Noise Ratio Light Spectroscopy of Tissue”) discloses a diagnostic system which delivers an optical beam to a nonlinear element that broadens a spectrum of the first optical beam to at least 10 nanometers through a nonlinear effect in the nonlinear element. U.S. patent application 20210038083 (Islam, Feb. 11, 2021, “Multi-Wavelength Wearable Device for Non-Invasive Blood Measurements in Tissue”) discloses a system for measuring one or more physiological parameters with a wearable device that includes a light source comprising a driver and semiconductor sources that generate an output optical light.

U.S. patent application publication 20090005692 (Intes et al., Jan. 1, 2009, “Optical Imaging Method for Tissue Characterization”) and U.S. Pat. No. 8,565,862 (Intes et al., Oct. 22, 2013, “Optical Imaging Method for Tissue Characterization”) disclose a method for detecting and characterizing abnormalities within biological tissue by characterizing optical properties of the tissue. U.S. patent application publication 20180070891 (Jepsen, Mar. 15, 2018, “Imaging With Infrared Imaging Signals”) discloses using an infrared imaging signal to image tissue. U.S. patent application publication 20180335753 (Jepsen et al., Nov. 22, 2018, “Co-Located Imaging and Display Pixel”) discloses an optical transformation engine coupled between an image pixel and a display pixel. U.S. patent application publication 20190072897 (Jepsen et al., Mar. 7, 2019, “Applications of Diffuse Medium Imaging”) discloses methods and an apparatus for imaging translucent materials.

U.S. Pat. No. 9,314,218 (Stearns et al., Apr. 19, 2016, “Integrated Microtomography and Optical Imaging Systems”) and U.S. Pat No. 10,130,318 (Stearns et al., Nov. 20, 2018, “Integrated Microtomography and Optical Imaging Systems”) disclose an integrated microtomography and optical imaging system with a rotating table that supports an imaging object, an optical stage, and separate optical and microtomography imaging systems. U.S. Pat. No. 9,770,220 (Stearns et al., Sep. 26, 2017, “Integrated Microtomography and Optical Imaging Systems”) discloses a rotating table that supports an imaging object, an optical stage, and separate optical and microtomography imaging systems. U.S. patent application 20170209083 (Zarandi et al., 2017, “Hand-Held Optical Scanner for Real-Time Imaging of Body Composition and Metabolism”) and U.S. Pat. No. 10,653,346 (Zarandi et al., May 19, 2020, “Hand-Held Optical Scanner for Real-Time Imaging of Body Composition and Metabolism”) disclose a handheld system for diffuse optical spectroscopic imaging of human tissue.

U.S. patent application 20060173352 (Lilge et al., 2006, “Optical Transillumination and Reflectance Spectroscopy to Quantify Disease Risk”) discloses a method of illuminating tissue of a mammal with light having wavelengths covering a pre-selected spectral range, detecting light transmitted through, or reflected from, the volume of selected tissue, and obtaining a spectrum of the detected light. U.S. patent application 20200116630 (Zhu, 2020, “Compact Guided Diffuse Optical Tomography System for Imaging a Lesion Region”) discloses a compact diffuse optical tomography system with laser diodes and a laser diode driver board. U.S. Pat. No. 5,876,339 (Lemire, Mar. 2, 1999, “Apparatus for Optical Breast Imaging”) discloses an optical breast imager with an adjustable volume which encloses a patient's breast.

U.S. Pat No. 5,999,836 (Nelson et al., Dec. 7, 1999, “Enhanced High Resolution Breast Imaging Device and Method Utilizing Non-Ionizing Radiation of Narrow Spectral Bandwidth”) and U.S. Pat. No. 6,345,194 (Nelson et al., Feb. 5, 2002, “Enhanced High Resolution Breast Imaging Device and Method Utilizing Non-Ionizing Radiation of Narrow Spectral Bandwidth”) disclose breast imaging using collimated non-ionizing radiation in the near ultraviolet, visible, infrared, and microwave regions. U.S. Pat. No. 6,240,309 (Yamashita et al., May 29, 2001, “Optical Measurement Instrument for Living Body”), U.S. Pat. No. 6,640,133 (Yamashita et al., Oct. 28, 2003, “Optical Measurement Instrument for Living Body”), and U.S. Pat. No. 7,142,906 (Yamashita et al., Nov. 28, 2006, “Optical Measurement Instrument for Living Body”) disclose an optical measurement instrument which applies visible-infrared light to several positions on a patient.

U.S. patent application 20020045833 (Wake et al., Apr. 18, 2002, “Medical Optical Imaging Scanner Using Multiple Wavelength Simultaneous Data Acquisition for Breast Imaging”) discloses a scanner for a medical optical imaging device with an illumination source which directs emitted light into a breast positioned below a support surface. U.S. Pat. No. 6,571,116 (Wake et al., May 27, 2003, “Medical Optical Imaging Scanner Using Multiple Wavelength Simultaneous Data Acquisition for Breast Imaging”) and U.S. Pat. No. 6,738,658 (Wake et al., May 18, 2004, “Medical Optical Imaging Scanner Using Multiple Wavelength Simultaneous Data Acquisition for Breast Imaging”) disclose a medical optical imaging device with an illumination source that directs emitted light into a breast positioned below a support surface.

U.S. patent application publication 20040092826 (Corbeil et al., May 13, 2004, “Method and Apparatus for Optical Imaging”) and U.S. Pat. No. 7,809,422 (Corbeil et al., Oct. 5, 2010, “Method and Apparatus for Optical Imaging”) disclose a platform with a cavity into which one of the person's breasts is suspended for optical imaging. U.S. patent application publication 20070287897 (Faris, Dec. 13, 2007, “Optical Vascular Function Imaging System and Method for Detection and Diagnosis of Cancerous Tumors”) discloses an in-vivo optical imaging system and method of identifying unusual vasculature associated with tumors. U.S. Pat. No. 8,027,711 (Jones et al., Sep. 27, 2011, “Laser Imaging Apparatus with Variable Patient Positioning”) discloses a tabletop to support a patient in front-down position and an opening to permit a breast of the patient to be vertically pendant below the tabletop.

U.S. Pat. No. 8,224,426 (Lilge et al., Jul. 17, 2012, “Optical Transillumination and Reflectance Spectroscopy to Quantify Disease Risk”) discloses spectroscopic tissue volume measurements with non-ionizing radiation to detect pre-disease transformations in tissue. U.S. patent application publication 20160066811 (Mohamadi, Mar. 10, 2016, “Handheld and Portable Scanners for Millimeter Wave Mammography and Instant Mammography Imaging”) discloses an array of ultra-wide band radio frequency sensors for breast imaging. U.S. Pat. No. 9,513,276 (Tearney et al. , Dec. 6, 2016, “Method and Apparatus for Optical Imaging via Spectral Encoding”) disclose a method, apparatus and arrangement for obtaining information associated with a sample such as a portion of an anatomical structure. U.S. patent application publication 20170007187 (Breneisen et al., Jan. 12, 2017, “Cancer Detector Using Deep Optical Scanning”) discloses Deep Optical Scanning (DEOS) for the detection of breast cancer and the determination of response to therapy.

U.S. Pat. No. 9,597,046 (Goossen et al., Mar. 21, 2017, “Method and Device for Imaging Soft Body Tissue Using X-Ray Projection and Optical Tomography”) discloses breast imaging using X-ray projection techniques and optical tomography techniques. U.S. patent application 20170105625 (Eum, Apr. 20, 2017, “Diagnostic Device of Optics Type for Breast”) discloses an optical breast diagnostic apparatus with a hemispherical cover. U.S. Pat. No. 10,200,655 (Kim et al., Feb. 5, 2019, “Tomographic Imaging Methods, Devices, and Systems”) discloses a multispectral bioluminescence optical tomography algorithm makes use of a partial differential equation (PDE) constrained approach. U.S. Pat. No. 10,215,636 (Fujii et al., Feb. 26, 2019, “Imaging Device Provided With Light Source That Emits Pulsed Light and Image Sensor”) discloses an imaging device with a light source that emits pulsed light at different wavelengths. U.S. Pat. No. 10,506,181 (Delgado et al., Dec. 10, 2019, “Device for Optical Imaging”) discloses the capture of an infrared image.

### SUMMARY OF THE INVENTION

A smart bra with optical sensors can be used to detect, image, and evaluate abnormal breast tissue. A cup on the smart bra can include a proximal outer ring, expandable chambers inside the proximal outer ring, and light emitters and light receivers between the expandable chambers and a person's breast. Changes in light emitted from the light emitters and received by the light receivers which are caused by transmission of the light through breast tissue can be analyzed to detect, image, and evaluate abnormal breast tissue.

### BRIEF INTRODUCTION TO THE FIGURES

FIGS. 1 and 2 show two views of a smart bra cup with inner and outer proximal rings, and crescent-shaped expandable chambers and optical sensors on either side of the cup.

FIGS. 3 and 4 show two views of a smart bra cup with inner and outer proximal rings, and a plurality of expandable chambers and optical sensors on either side of the cup.

FIGS. 5 and 6 show two views of a smart bra cup with an outer proximal rings, and a plurality of expandable chambers and optical sensors on either side of the cup.

FIG. 7 shows a frontal view of a bra and a close-up view of one of the bra cups (including rings, expandable chambers, and optical sensors) wherein a longitudinal axis of an inner ring is parallel to a bustline vector.

FIG. 8 shows a frontal view of a bra and a close-up view of one of the bra cups (including rings, expandable chambers, and optical sensors) wherein a longitudinal axis of an inner ring is oblique relative to a bustline vector.

### DETAILED DESCRIPTION OF THE FIGURES

With respect to current methods for detecting abnormal breast tissue, x-ray mammography causes exposure to ionizing radiation and is not very mobile, MRI is expensive and not very mobile, and ultrasound can have low sensitivity and be labor intensive. A smart bra with optical sensors can provide (supplemental) screening for abnormal breast tissue without exposure to ionizing radiation, especially in areas with poor geographic access to large radiological equipment. In an example, a smart bra with optical sensors can be used to periodically (e.g. daily, weekly, monthly, or annually) scan a person's breast to measure changes over time in tissue density within the breast.

In an example, a smart bra cup can comprise: a proximal outer ring which is part of a smart bra cup, wherein the outer ring is configured to encircle a person's breast; a plurality of expandable chambers inside the proximal outer ring; and a plurality of light emitters and light receivers between the plurality of expandable chambers and the person's breast; wherein changes in light emitted from the light emitters and received by the light receivers which are caused by transmission of the light through breast tissue are analyzed to detect, locate, evaluate, characterize, and/or image abnormal breast tissue.

In an example, a proximal outer ring can have a dorsal-to-ventral width between 1 and 3 inches. In an example, a proximal outer ring can span between 20% and 70% of the dorsal-to-ventral depth of a smart bra cup. In an example, a proximal outer ring can have an annular shape. In an example, a proximal outer ring can have a frustal shape. In an example, an expandable chamber can have a crescent and/or banana shape.

In an example, a bra cup can further comprise a proximal inner ring which is inside a proximal outer ring. In an example, a longitudinal axis of a proximal inner ring can be parallel to a bustline vector, wherein the bustline vector is a line which connects the apexes of right side and left side cups of a bra. In an example, a longitudinal axis of a proximal inner ring can intersect a line which is parallel to a bustline vector at an angle between 30 and 60 degrees, wherein the bustline vector is a line which connects the apexes of right side and left side cups of a bra. In an example, there can be multiple expandable chambers in each of two spaces between a proximal inner ring and a proximal outer ring.

In an example, a smart bra cup can comprise: a proximal outer ring which is part of a smart bra cup, wherein the outer ring is configured to encircle a person's breast; a proximal inner ring which is part of the smart bra cup, wherein the inner ring is configured to encircle the person's breast and wherein the inner ring is inside the outer ring; a plurality of expandable chambers between the inner ring and the outer ring; an elastic concave distal portion of the smart bra cup which covers a distal portion of the person's breast; and a plurality of light emitters and light receivers between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

In an example, a proximal outer ring can have a dorsal-to-ventral width between 1 and 3 inches. In an example, a proximal outer ring can span between 20% and 70% of the dorsal-to-ventral depth of a smart bra cup. In an example, a proximal outer ring can have an annular shape. In an example, a proximal outer ring can have a frustal shape. In an example, a longitudinal axis of a proximal inner ring can be parallel to a bustline vector, wherein the bustline vector is a line which connects the apexes of right side and left side cups of a bra. In an example, a longitudinal axis of a proximal inner ring can intersect a line which is parallel to a bustline vector at an angle between 30 and 60 degrees, wherein the bustline vector is a line which connects the apexes of right side and left side cups of a bra. In an example, an expandable chamber can have a crescent and/or banana shape. In an example, there can be multiple expandable chambers in each of two spaces between a proximal inner ring and a proximal outer ring.

In an example, a smart cup insert can comprise: a cup insert which is configured to be removably inserted between the cup of a bra and a person's breast; wherein the cup insert further comprises a plurality of expandable chambers; and wherein the cup insert further comprises a plurality of light emitters and light receivers; and wherein changes in light emitted from the light emitters and received by the light receivers which are caused by transmission of the light through breast tissue are analyzed to detect, locate, evaluate, characterize, and/or image abnormal breast tissue.

In an example, a smart bra cup with optical sensors can be used to measure the vascular dynamics of a breast. In an example, analysis of how near infrared light from light emitters in a smart bra is changed by transmission through breast tissue can be used to measure levels and/or concentration locations of deoxygenated hemoglobin in breast tissue. In another example, analysis of how red and near infrared light from light emitters in a smart bra is changed by transmission through breast tissue can be used to measure levels and/or concentration locations of oxygenated hemoglobin in breast tissue.

In an example, light which has been transmitted from a light emitter to a light receiver through breast tissue can be analyzed to identify the levels, concentrations, and/or locations of specific biological substances (e.g. markers) which are associated with abnormal breast tissue. In an example, light which has been transmitted through and/or reflected from breast tissue can be analyzed to detect and/or image levels, concentrations, and/or locations of hemoglobin in breast tissue. In an example, light which has been transmitted through and/or reflected from breast tissue can be analyzed to detect and/or image levels, concentrations, and/or locations of oxygenated hemoglobin in breast tissue. In one embodiment, light which has been transmitted through and/or reflected from breast tissue can be analyzed to detect and/or image the locations, sizes, and/or configurations of extracellular matrix structures in breast tissue.

In an example, changes in the amplitude and/or spectrum of light caused by transmission of the light through breast tissue can be analyzed to detect abnormal breast tissue (e.g. breast cancer) and/or create an image of breast tissue. In an example, joint three-dimensional analysis of light transmitted through multiple intersecting vectors between multiple pairs of light emitters and light detectors can increase the accuracy of identification and location of abnormal tissue.

In an example, changes in the amplitude and/or intensity of light caused by transmission through breast tissue can be analyzed to identify the presence, composition, shape, size, and/or location of abnormal breast tissue. In an example, changes in the frequency of light caused by transmission through breast tissue can be analyzed to identify the presence, composition, shape, size, and/or location of abnormal breast tissue. In another example, light beams which have been sequential transmitted between different pairs of light emitters and light receivers, along different vectors through breast tissue, can be analyzed to identify the presence, composition, shape, size, and/or location of abnormal breast tissue. In an example, the amplitude, intensity, and/or brightness of light from a light emitter in a smart bra can be automatically adjusted based on analysis of light received from a light receiver in the smart bra.

In an example, light which has been transmitted from a light emitter to a light receiver through breast tissue can be analyzed using artificial intelligence and/or machine learning. In one embodiment, light which has been transmitted from a light emitter to a light receiver through breast tissue can be analyzed using Diffuse Optical Imaging (DOI). In an example, light which has been transmitted from a light emitter to a light receiver through breast tissue can be analyzed using Near Infrared Spectroscopy (NIRS). In an example, right and left slide cups of a smart bra can both have optical sensors (e.g. light emitters and light receivers) so that characteristics of a person's right and left side breasts can be compared (and contrasted) as part of detecting abnormal breast tissue.

In an example, the layer of a smart bra cup which is closest to a person's breast can be made with thermoelastic polymer. In another example, an optical smart bra can have a rigid frame. In an example, the frame for a cup in an optical smart bra can comprise four segments: a lower upward-opening concave partial-annular segment; an upper upward-opening concave segment; and two elastic segments which connect the ends of the lower and upper partial-annular segments. In another example, the frame for a cup in an optical smart bra can comprise two rigid partial-annular segments which are connected to each other by two elastic segments. In an example, a proximal outer ring of a smart bra cup can be relatively rigid (e.g. more rigid than the rest of the bra cup). In an example, the base of a smart bra cup (e.g. closest to the chest wall) can be less flexible, less elastic, and/or more rigid than the rest of the cup.

In an example, a smart bra can differ from a conventional bra in that it includes an arm sleeve portion in order to better scan the Tail of Spence. In an example, a smart bra cup can comprise: a lower-outer crescent-shaped portion on a lower-outer half of the cup; and a upper-inner crescent-shaped portion on a upper-inner half of the cup; wherein the lower-outer portion further comprises a lower-outer rigid outer layer, a lower-outer expandable chamber layer, and a lower-outer inner optical layer (with light emitters, light receivers, or both), wherein the inner layer is closest to a breast when the cup is worn; and wherein the upper-inner portion further comprises a upper-inner rigid outer layer, a upper-inner expandable chamber layer, and a upper-inner inner optical layer (with light emitters, light receivers, or both), wherein the inner layer is closest to a breast when the cup is worn.

In an example, a smart bra cup can have an elliptical shape (e.g. outer-perimeter or frame shape), wherein longitudinal axis of the shape spans a breast and the Tail of Spence along an oblique (lower-inner to upper-outer (vector). In another example, a smart bra frame to which a cup is attached can have an equestrian stirrup shape wherein the opening of the stirrup shape is oriented toward a person's armpit and/or shoulder (in order to better scan the Tail of Spence). In an example, the cross-sectional perimeter of a proximal outer ring can have a circular, oblate-circular, elliptical, oval, or teardrop shape. In another example, the outer surface of a smart bra cup can have a hemispherical shape.

In an example, a smart bra can comprise a plurality of light emitters which emit light at different wavelengths. In another example, light emitters in a smart bra can include a first set of light emitters which emit red light (e.g. approximately 660 nm) and a second set of light emitters which emit near infrared light (e.g. approximately 940 nm). In an example, light emitters in a smart bra can include: light emitters which emit green light (e.g. approximately 530 nm); light emitters which emit yellow or amber light (e.g. approximately 590 nm), light emitters which emit red light (e.g. approximately 660 nm); and light emitters which emit near infrared light (e.g. approximately 940 nm).

In an example, light emitters in a smart bra can be connected to fabric by laser microwelding. In one embodiment, light emitters in a smart bra can be connected to fabric by welding. In an example, light emitters in a smart bra can be connected via silver-plated nylon thread or yarn. In one embodiment, a light emitter in a smart bra can be encapsulated in a clear resin to protect against damage when the bra is washed. In another example, a light receiver (e.g. photodetector) can be encapsulated by placing the emitter in a mold, pouring a liquid and breathable polymer (e.g. porous PDMS) over it, and then curing the polymer (e.g. with heat and/or light). In an example, light emitters (e.g. LEDs) in an smart bra can be encapsulated in a transparent polymer for protection from damage when the bra is washed. In another example, light receivers (e.g. photodetectors) in an smart bra can be encapsulated in an elastomeric transparent polymer for protection from damage when the bra is washed.

In an example, a smart bra cup can comprise light emitters and light detectors on either side of an oblique virtual plane which spans between a upper-outer quadrant of a breast and a lower-inner quadrant of the breast. In an example, light emitters can be on a first side of a smart bra cup and light receivers can be on the opposite side of the cup. In an example, light emitters can be on a right side of a smart bra cup and light receivers can be on a left side of the cup, wherein light from the light emitters is transmitted through breast tissue before being received by the light receivers. In an example, a smart bra cup can include a concave array of light receivers. In an example, light emitters and receivers in a smart bra cup can be configured in a helix (e.g. from the base of the cup to the center and/or apex of the cup). In another example, light emitters and/or receivers in a smart bra cup can be configured in a helical array. In an example, light emitters and/or receivers in a smart bra cup can be configured in a star-burst array. In another example, light emitters in a smart bra cup can be configured in a helix (e.g. from the base of the cup to the center and/or apex of the cup).

In an example, a smart bra cup can comprise a beehive configuration with four rings of light emitters and/or detectors around a person's breast. In another example, a smart bra cup can comprise a stack of four rings of light emitters and/or detectors around a person's breast. In an example, a smart bra cup can comprise a stack of three rings of light emitters and/or detectors around a person's breast. In one embodiment, a smart bra cup can comprise three nested (e.g. concentric) rings of light emitters and/or detectors around a person's breast. In an example, light emitters (e.g. LEDs) and light receivers (e.g. photodetectors) in a cup of smart bra can be configured in a dorsal-to-ventral sequence of nested (e.g. concentric) rings of light emitters and receivers on the cup. In an example, light emitters (e.g. LEDs) in a cup of smart bra can be configured in a dorsal-to-ventral sequence of nested (e.g. concentric) rings of light emitters on the cup. In an example, there can be rings of optical sensors around the circumference of a cup, wherein each ring further comprises a sequence of different-color light emitters and light receivers.

In an example, distances between light emitters and light receivers on a smart bra cup can be greater for proximal (e.g. closer to the chest wall) portions of the cup than for distal (e.g. farther from the chest wall) portions of the cup. In an example, the density of light emitters and/or light receivers can be greater in proximal portions of a smart bra cup than in distal portions of the cup. In another example, the density of light emitters in a smart bra cup can be greater for portions of the cup which farther from the chest wall and less for portions of the cup which are closer to the chest wall. In an example, the density of light receivers in a smart bra cup can be greater for portions of the cup which closer to the chest wall and less for portions of the cup which are farther from the chest wall.

In an example, the density of light receivers in the upper-outer quadrant of a smart bra cup can be less than in the lower-inner quadrant of the cup. In an example, the density of optical sensors in a smart bra cup can be greater for portions of the cup which closer to the chest wall and less for portions of the cup which are farther from the chest wall. In an example, the density of optical sensors in the upper-outer quadrant of a smart bra cup can be less than in the lower-inner quadrant of the cup. In one embodiment, the angles between the surface of a smart bra cup and light beams emitted from light emitters toward the surface of a breast can increase with the distance of the light emitters from the apex of the cup.

In an example, a light emitter in a smart bra scan emit light at different wavelengths at different times, ranging from green light (e.g. approximately 530 nm) to yellow or amber light (e.g. approximately 590 nm) to red light (e.g. approximately 660 nm) to near infrared light (e.g. approximately 940 nm). In another example, a smart bra can comprise a light emitter which emits light at different angles and/or vectors at different times. In one embodiment, a smart bra can comprise a plurality of light emitters which emit light at different times. In another example, LEDs emitting red light and LEDs emitting near infrared light in a smart bra can be flashed, pulsed, and/or activated in an alternating and repeating (e.g. red, near infrared, neither, red, near infrared, neither, etc.) sequence.

In an example, a smart bra cup can comprise: a convex (e.g. circular or elliptical) transparent tube which encircles a person's breast; one or more light emitters and/or light receivers inside the tube which move around (a portion of) the circumference of the tube. In another example, a smart bra can comprise a plurality of actuators which move light receivers in different directions or to different locations on a cup. In an example, a smart bra cup can comprise: a convex (e.g. circular or elliptical) transparent tube which encircles a person's breast; one or more light emitters and/or light receivers inside the tube; and one or more actuators which move the light emitters and/or light receivers around (a portion of) the circumference of the tube. In an example, a smart bra cup can comprise a plurality of optical sensor modules, wherein each module further comprises at least one light emitter, a movable light guide (e.g. movable micromirror array), and at least one light receiver. In an example, the vector long which a light emitter on a smart bra cup emits light into a breast can be moved, adjusted, and/or controlled by changing an electrical field which moves a light guide (e.g. micromirror) which redirects light from the light emitter.

In an example, a smart bra cup can comprise a plurality of optical sensor modules, wherein each module further comprises a light emitters and two light receivers. In an example, a smart bra cup can comprise a plurality of optical sensor modules, wherein each module further comprises a plurality of light emitters and a light receiver. In another example, a smart bra cup can comprise a plurality of optical sensor modules, wherein each module further comprises two light emitters and a light receiver. In an example, a smart bra can comprise a plurality of optical fibers which transmit light from light emitters to different locations on the interior surface of a cup. In another example, a smart bra can include polymer optical fibers which are woven into the fabric used to make the bra.

In an example, expansion of expandable chambers can gently compress a person's breast for more accurate optical scanning over shorter distances. In one embodiment, an expandable chamber can be an air-tight compartment or pocket. In an example, a smart bra cup can include a plurality of expandable chambers which are expanded by being filled with a liquid. In an example, an expandable chamber can be expanded by being filled with a liquid (e.g. saline solution).

In an example, a smart bra can include a manual pump which is operated by the person wearing the bra, wherein the manual pump sends a flowable substance (e.g. air or saline) into expandable chambers in a smart bra cup. In an example, a smart bra can include a plurality of tubes which conduct a flowable substance from a pump to a plurality of expandable chambers in a cup, wherein there is a separate tube for each chamber in order to enable selective and differential expansion of individual chambers. In another example, the flow rate of a flowable substance into an expandable chamber in a smart bra cup can vary over time, wherein the flow rate start out at a higher rate when a pump is first activated and decreases to a lower rate as time after activation increases.

In an example, a person wearing a smart bra can actively control the operation of a pump so that it can be stopped (or reversed) if compression of the person's breast becomes uncomfortable. In another example, a smart bra cup can gently compress a person's breast by controlled expansion of a plurality of expandable (e.g. inflatable) chambers, wherein expansion (e.g. inflation) of the chambers is controlled by the person wearing the bra.

In an example, a smart bra cup can comprise an array of individually-controllable expandable chambers, wherein selective and differential expansion of chambers in this array enable customizing the shape of the cup to the size and shape of a specific breast. In another example, expandable chambers in a smart bra cup can be individually and selectively expanded by different degrees, by different amounts, to different sizes, and/or to different internal pressures, thereby exerting different levels of pressure on different portions of a breast. In an example, a smart bra can comprise a plurality of expandable chambers which are expanded by different amounts. In an example, the flow rate of a flowable substance into an expandable chamber in a smart bra cup can be non-uniform over time. In one embodiment, a smart bra can comprise a plurality of expandable chambers which are expanded at different times, wherein chambers that are closer to the bra cup center are expanded after chambers which are farther from the bra cup center. In an example, expandable chambers in a sequence of expandable chambers in a smart bra cup can be sequentially-expanded at different times to create a compression wave which compresses a person's breast in the cup.

In an example, a smart bra cup can comprise a first concave expandable chamber on a lower-inner half of the cup and a second concave expandable chamber on an upper-outer half of the cup, wherein one end of the second concave expandable chamber spans the Tail of Spence. In another example, a smart bra cup can comprise a first expandable chamber on a first side of the cup and a second expandable chamber on a second side of the cup, wherein the second side is opposite the first side. In an example, a smart bra cup can gently compress a person's breast by controlled expansion of a plurality of expandable (e.g. inflatable) chambers on each of the upper and lower halves of the cup (when the person's is standing). In another example, a smart bra cup can include an expandable chamber (e.g. a chamber which is expanded by being filled with a flowable substance) in the lower half of the cup, wherein the cross-section of the expandable chamber has a concave shape.

In an example, a first expandable chamber in a smart bra cup which is closer to the center of the cup can be larger than a second expandable chamber in the smart bra cup which is farther from the center of the cup. In another example, a lower half of a smart bra cup can have smaller expandable chambers than an upper half of the cup. In an example, a smart bra cup can comprise a stacked array of annular expandable chambers which encircles a breast, wherein chambers which are closer to the chest wall are larger and/or wider than chambers which are farther from the chest wall. In an example, a lower half of a smart bra cup can have fewer expandable chambers than an upper half of the cup.

In an example, a smart bra cup can comprise a nested array of annular (e.g. toroidal) expandable chambers which encircle a breast. In an example, a smart bra cup can comprise a plurality of longitudinal (e.g. cylindrical) expandable (e.g. inflatable) chambers in the lower half of the cup which lift and gently compress a breast when the chambers are expanded. In another example, a smart bra cup can comprise a stacked array of annular expandable chambers which encircles a breast. In one embodiment, a smart bra cup can comprise an annular array (e.g. ring) of expandable chambers which encircle the base on a person's breast on the chest wall, wherein the forward-central portion of the cup is open to allow the breast to extend forward during expansion. In another example, an expandable chamber can be shaped like a quarter of a circle, ring, or torus. In an example, an expandable chamber in a smart bra cup can be expanded into a keystone, trapezoidal, or annular section shape. In one embodiment, an expandable chamber in a smart bra cup can have an annular and/or toroidal shape. In an example, there can be multiple expandable chambers in each of two spaces (e.g. gaps) between a proximal inner ring and a proximal outer ring in a smart bra cup.

In an example, a (crescent-shaped or elliptical) expandable chamber in a smart bra cup can have a longitudinal axis which intersects a line parallel to the bustline vector of the bra at an angle between 5 and 25 degrees, wherein the bustline vector is defined as a horizontal line that connects the apexes of the right and left cups of the bra. In an example, a smart bra cup can comprise a first crescent-shaped expandable chamber on a lower-inner half of the cup and a second crescent-shaped expandable chamber on an upper-outer half of the cup.

In an example, a smart bra cup can comprise: a (frustum shaped) rigid outer ring around a person's breast close to the chest wall; a concave array of optical components (light emitters and/or light receivers) around the person's breast; and two crescent-shaped expandable chambers between the rigid outer ring and the concave array of optical components, wherein the expandable chambers are expanded by being filled with a flowable substance (e.g. a gas, fluid, or gel), wherein the expandable chambers combine to span between 70% and 95% of a circumference of the cup, wherein a first crescent-shaped expandable chamber is on a first half of the cup, and wherein a second crescent-shape expandable chamber is on a second half of the cup which is opposite the first half.

In an example, a smart bra cup can comprise: a frustum-shaped rigid outer ring around a person's breast close to the chest wall; a concave array of light emitters and/or light receivers around the person's breast; and two or more crescent-shaped expandable chambers between the rigid outer ring and the concave array, wherein the expandable chambers are expanded by being filled with a flowable substance (e.g. a gas, fluid, or gel), wherein a first crescent-shaped expandable chamber is on the lower half of the cup, and wherein a second crescent-shape expandable chamber is on the upper half of the cup. In an example, a smart bra cup can include a plurality of expandable chambers (e.g. chambers which are expanded by being filled with a flowable substance) in the lower half of the cup, wherein the combined cross-section of the plurality of expandable chambers has a crescent shape. In another example, an expandable chamber in a smart bra cup can be expanded into an oblong, crescent, and/or banana shape. In an example, an expandable chamber in a smart bra cup can have an upwardly-concave crescent shape.

In an example, a plurality of expandable chambers can be formed from two polymer layers, wherein chambers (e.g. indentations, bubbles, or pockets) are created in a first polymer layer by moving the layer over a mold and applying negative pressure is selected locations when the first layer is still hot, and wherein a second polymer is adhered to the first layer to seal the chambers. In an example, a smart bra cup can include a layer of expandable chambers and optical elements (e.g. light emitters and/or receivers), wherein there is sequence which alternates between expandable chambers and optical elements around a circumference of the cup and/or breast.

In an example, a proximal inner ring on a smart bra cup can be less circular than a proximal outer ring on the cup. In an example, a smart bra cup can comprise: (a) an outer frustum-shaped layer around a person's breast, wherein the outer layer is a first average distance from the person's breast; (b) an inner frustum-shaped layer around the person's breast, wherein the inner layer is a second average distance from the person's breast, wherein the second average distance is less than the first average distance; (c) a plurality of expandable components between the inner layer and the outer layer; and (d) a plurality of optical components (light emitters and/or light receivers) around the person's breast between the inner layer and the person's breast. In one embodiment, instead of a continuous inner ring, a smart bra cup can have two arcuate inner segments (e.g. one arcuate segment on either side of the cup). In an example, a first portion of an expandable chamber in a smart bra cup which is closer to the surface of a person's breast can be narrower than a second portion of the chamber in the smart bra cup which is farther from the person's breast. In another example, a proximal outer ring of a smart bra cup can span between 20% and 70% of the dorsal-to-ventral depth of the cup.

In an example, a smart bra cup can comprise: a first arcuate section (or layer) which is configured to be a first distance from the surface of a person's breast; a second arcuate section (or layer) which is configured to be a second distance from the surface of the person's breast, wherein the second distance is greater than the first distance; a plurality of expandable chambers between the first arcuate section (or layer) and the second arcuate section (or layer), wherein expansion of the expandable chambers gently compresses the breast by pushing the first arcuate section (or layer) toward the breast; and a plurality of elastic sections (or bands) between the expandable chambers, wherein the elastic sections connect the first arcuate section (or layer) and the second arcuate section (or layer).

In an example, a smart bra cup can comprise: a first portion on a first half of the cup; a second portion on a second half of the cup; and elastic third and fourth portions which connect the first and second portions to each other; wherein the first portion further comprises a first rigid outer layer, a first expandable chamber layer, and a first inner optical layer (with light emitters, light receivers, or both), wherein the inner layer is closest to a breast when the cup is worn; and wherein the second portion further comprises a second rigid outer layer, a second expandable chamber layer, and a second inner optical layer (with light emitters, light receivers, or both), wherein the inner layer is closest to a breast when the cup is worn.

In an example, a smart bra can comprise light emitters, light receivers, and electroconductive pathways which are printed on an elastomeric substrate. In another example, a smart bra cup can comprise a deformable transparent gel layer between optical components (e.g. light emitters and/or receivers) and the surface of a person's breast. In an example, a smart bra cup can include an inner layer of opaque elastomeric polymer (e.g. PDMS which has been dyed black), wherein there is an array of holes and/or openings in this inner layer, and wherein there are optical elements (e.g. light emitters and/or receivers) in the holes and/or openings.

In an example, the inner-most layer of a smart bra cup can be between 0.5 mm and 3 mm thick. In an example, the innermost layer of a smart bra cup can be made from a transparent elastomeric material. In an example, the inner-most layer of a smart bra cup can be opaque. In one embodiment, the innermost layer of a smart bra cup can comprise a 1 mm to 5 mm elastomeric polymer layer. In an example, the innermost layer of a smart bra cup can comprise an elastomeric polymer (e.g. PDMS). In another example, the innermost layer of a smart bra cup can comprise an elastomeric polymer. In one embodiment, the innermost layer of a smart bra cup can comprise an opaque elastomeric polymer layer with openings (e.g. holes) for light emitters and light receivers. In another example, the inner-most layer of a smart bra cup can have a Shore durometer level which is less than 30. In an example, the inner-most layer of a smart bra cup can have an optical absorption coefficient with a value like that of normal breast tissue. In another example, the layer of a smart bra cup which is closest to a person's breast can be made with an opaque (e.g. dyed black) silicone-based polymer.

In an example, a distal concave portion of a smart bra cup can have a hemispherical, hemi-ellipsoidal, half oblate-spheroidal, or paraboloidal shape. In an example, a smart bra cup can be annular with a frontal opening through which a person's breast can extend forward during gentile compression by expandable chambers. In an example, the front portion of a smart bra cup can be open, allowing a person's breast to extend forward during gentle compression of the breast by expandable chambers. In an example, the longitudinal axis of a proximal inner ring in a smart bra cup can be parallel to a bustline vector connecting the apexes of right side and left side bra cups.

In an example, a smart bra can include a plurality of electroconductive elastomeric polymer (e.g. PDMS which has been doped or impregnated with conductive material) pathways which transmit electricity to light emitters (e.g. LEDs). In an example, a smart bra can include electroconductive pathways which are created by printing conductive and elastic ink onto the fabric of the bra. In another example, a smart bra can include undulating (e.g. sinusoidal, serpentine or zigzag) electroconductive thread which transmits electrical signals from light receivers (e.g. photodetectors) on a bra frame. In an example, a smart bra can include undulating (e.g. sinusoidal, serpentine or zigzag) insulated wires which transmit electrical signals from light receivers (e.g. photodetectors).

In an example, a smart bra cup can comprise one or more elastic electroconductive pathways which are configured in a hub-and-spoke pattern. In an example, a smart bra cup can comprise one or more elastic electroconductive pathways which are configured in a spiral or helical pattern. In another example, a smart bra cup can include a plurality of elastic electroconductive pathways which provide electrical connections among light emitters, light receivers, and a data processor on the bra. In one embodiment, a smart bra cup can include undulating wires, conductive threads, or conductive yarns which transmit electrical power to light emitters. In an example, electroconductive pathways on a smart bra can be formed by printing ink onto the fabric of the bra, wherein the ink is a mixture of conductive and adhesive materials.

In an example, a first expandable chamber in a smart bra cup which is closer to the center of the cup can be filled with a flowable substance to a lesser interior pressure level than a second expandable chamber in the smart bra cup which is farther from the center of the cup. In an example, a smart bra can comprise a plurality of expandable chambers which are expanded to different interior pressure levels, wherein chambers that are closer to the bra cup center are expanded to higher pressure levels than chambers which are farther from the bra cup center. In an example, a smart bra can include a compression-monitoring sensor which monitors pressure exerted by the interior of a cup on a person's breast, wherein the compression-monitoring sensor is a pressure sensor.

In an example, a smart bra cup can include a motion and/or bend sensor which measures the amount by which expansion of one or more expandable chambers compresses a breast within the cup. In an example, data from a pressure sensor can be analyzed to limit (or otherwise control) the expansion of an expandable chamber in a smart bra. In another example, the flow rate of a flowable substance which is pumped into an expandable chamber in a smart bra cup can decrease as the pressure level between the cup and a person's breast within the cup increases. In an example, the operation of a pump on a smart bra can be controlled by a pressure sensor which is in fluid communication with the interior of an expandable chamber.

In an example, a smart bra cup can comprise a plurality of piezoelectric strands, strips, or straps which gently compress a breast when electrical energy is applied to them. In an example, a smart bra can include a plurality of hydraulic chambers or actuators. In an example, the frame of a smart bra cup can be gently and temporarily adhered to a person's chest wall, encompassing the Tail of Spence, the upper outer quadrant, the upper inner quadrant, the lower inner quadrant, and the lower outer quadrant of a breast. In an example, a wearable device for optical scanning of a person's breast to detect abnormal tissue can comprise a convex (e.g. circular or elliptical) transparent tube which is placed on the chest wall around the person's breast.

In an example, a bra insert with optical sensors can be rotated between multiple attachments and detachments to scan a breast from different orientations, angles, and/or locations. In one embodiment, a bra insert with optical sensors for detecting abnormal breast tissue can be inserted between the cup of a traditional bra and a person's breast within the cup. In another example, a smart bra insert with optical sensors can be removably attached to the concave interior of a conventional bra cup. In an example, a smart bra system can comprise two-part fasteners, wherein a first part of a two-part fastener is part of a removable bra insert with optical sensors, wherein a second part of the two-part fastener is attached to a bra cup, wherein this two-part fastener design allows the bra insert to be removed from the bra before the bra is washed, but also ensures that the bra insert is positioned in the same location on a breast for scans at different times (e.g. on the monthly or annual basis), enabling more-precise longitudinal analysis of changes in breast tissue for detection of abnormal breast tissue. In another example, a bra cup can have a pocket, pouch, or compartment into which optical sensors (and electronics) can be placed, wherein the optical sensors can be removed before the bra is washed and put back after the bra has been washed.

In an example, a smart bra can comprise an adhesive ring which is gently adhered to the chest wall where a breast is attached to the chest wall. In another example, a wearable device with optical sensors for detecting abnormal breast tissue can be embodied in a concave adhesive patch, stick, or bandage which is place on a person's breast. In an example, a wearable device with optical sensors for detecting abnormal breast tissue can be embodied in an annular adhesive patch, stick, or bandage which encircles a person's breast. In an example, a smart bra can further comprise a data processor. In an example, a smart bra can further comprise a wireless data transmitter. In an example, data from optical sensors on a smart bra can be wirelessly transmitted to a data processor in a different wearable device (e.g. a smart watch), a handheld device (e.g. a cell phone), or a remote server (e.g. in a healthcare provider's server and/or cloud storage).

In an example, a smart bra with optical sensors can be used to periodically (e.g. daily, weekly, monthly, or annually) scan a person's breast to measure changes over time in deoxyhemoglobin levels, concentrations, and/or locations within the breast. In an example, the effect of transmission through breast tissue on the amplitude and/or spectrum of light at a first time can be compared to the effect of transmission through the breast tissue on the amplitude and/or spectrum of light at a second time can be analyzed to identify abnormal breast tissue.

In an example, analysis of how light from light emitters in a smart bra is changed by transmission through breast tissue can be used to measure levels and/or concentration locations of deoxygenated hemoglobin in breast tissue. In an example, analysis of how near infrared light from light emitters in a smart bra is changed by transmission through breast tissue can be used to measure levels and/or concentration locations of oxygenated hemoglobin in breast tissue. In another example, changes in the amplitude and/or spectral distribution of light from light emitters on a smart bra which are caused by its transmission through a breast tissue can be analyzed to evaluate the molecular composition of breast tissue. In an example, light which has been transmitted through and/or reflected from breast tissue can be analyzed to detect and/or image levels, concentrations, and/or locations of collagen in breast tissue. In one embodiment, light which has been transmitted through and/or reflected from breast tissue can be analyzed to detect and/or image levels, concentrations, and/or locations of lipids in breast tissue. In an example, light which has been transmitted through and/or reflected from breast tissue can be analyzed to detect and/or image levels, concentrations, and/or locations of water in breast tissue.

In an example, changes in the amplitude and/or spectral distribution of light from light emitters on a smart bra which are caused by its transmission through a breast tissue can be analyzed to create a three-dimensional image of the breast. In an example, changes in the intensity and/or spectral distribution of light caused by transmission through breast tissue can be analyzed to create a 3D image of breast tissue. In another example, joint three-dimensional analysis of light transmitted through multiple intersecting vectors between multiple pairs of light emitters and light detectors can provide redundant data which increases the accuracy of identification and location of abnormal tissue.

In an example, changes in the amplitude and/or spectral distribution of light from light emitters on a smart bra which are caused by its transmission through a breast tissue can be analyzed to evaluate the size, shape, density, molecular composition, and/or location of abnormal breast tissue. In another example, changes in the spectral distribution of light caused by transmission through breast tissue can be analyzed to identify the presence, composition, shape, size, and/or location of abnormal breast tissue. In an example, light which has been transmitted between different pairs of light emitters and light receivers, along different vectors through breast tissue, can be analyzed to identify the presence, composition, shape, size, and/or location of abnormal breast tissue. In an example, the amplitude, intensity, and/or brightness of light from a light emitter in a smart bra can be automatically increased if the amount of light received from a light receiver in the smart bra is insufficient for accurate analysis.

In an example, light which has been transmitted from a light emitter to a light receiver through breast tissue can be analyzed using Carlavian Curve analysis (CCA). In an example, light which has been transmitted from a light emitter to a light receiver through breast tissue can be analyzed using Diffuse Optical Tomography (DOT). In an example, light which has been transmitted from a light emitter to a light receiver through breast tissue can be analyzed using spectroscopic analysis.

In an example, a smart bra can be made with a combination of elastane and nylon. In an example, a frame for a cup in an optical smart bra can comprise a longitudinal rigid-polymer piece which is enclosed in fabric. In another example, the frame for a cup in an optical smart bra can be rigid. In one embodiment, the frame for a cup in an optical smart bra can comprise four segments: two rigid partial-annular segments; and two elastic segments connecting the ends of the partial-annular segments. In an example, a distal concave portion (e.g. distal dome) of a smart bra cup can be more elastic, more flexible, and/or less rigid than other portions of the cup. In an example, portions of a smart bra cup which are closer to the apex of the cup can be more elastic, more flexible, and/or less rigid than other areas of the cup. In an example, the center of a smart bra cup can be more flexible, more elastic, and/or less rigid than the perimeter of the cup.

In an example, a smart bra cup can comprise: a first crescent-shaped portion on a first half of the cup; and a second crescent-shaped portion on a second half of the cup; wherein the first portion further comprises a first rigid outer layer, a first expandable chamber layer, and a first inner optical layer (with light emitters, light receivers, or both), wherein the inner layer is closest to a breast when the cup is worn; and wherein the second portion further comprises a second rigid outer layer, a second expandable chamber layer, and a second inner optical layer (with light emitters, light receivers, or both), wherein the inner layer is closest to a breast when the cup is worn. In an example, a smart bra cup can have a paisley shape (e.g. outer-perimeter or frame shape). In another example, a smart bra cup can span a Tail of Spence. In an example, an upper-outer quadrant of a perimeter of a smart bra cup can have an inward-facing concavity (e.g. opening toward the center of the cup) for better coverage of the Tail of Spence. In an example, the frame for a cup in an optical smart bra can have an annular shape.

In an example, a smart bra can include an arcuate light emitter strip and/or band (e.g. LED strip) on the inside of a bra cup. In an example, light emitters in a smart bra can be NIR OLEDs. In one embodiment, light emitters in a smart bra can include near-infrared (NIR) light emitters (e.g. LEDs) which emit light in range of 700 nm to 1400 nm. In an example, a light emitter can be attached to a flexible polymer film (e.g. polyimide or PET) and the film is then integrated into the fabric used to make a smart bra. In another example, light emitters in a smart bra can be connected to fabric by sewing or embroidering. In an example, light emitters in a smart bra can be connected via conductive thread or yarn. In another example, light emitters in a smart bra can be connected via thread or yarn with stainless steel strands.

In an example, a light emitter in a smart bra can be encapsulated in a nanolaminate film to protect it from damage when the bra is washed. In an example, a light receiver (e.g. photodetector) can be encapsulated by placing the emitter in a mold, pouring a liquid transparent polymer (e.g. PDMS) over it, and then curing the polymer (e.g. with heat and/or light). In an example, light emitters (e.g. LEDs) in an smart bra can be encapsulated in an elastomeric transparent polymer for protection from damage when the bra is washed. In an example, light receivers (e.g. photodetectors) in an smart bra can be encapsulated in a transparent polymer for protection from damage when the bra is washed.

In an example, light emitters can be on a first half of a smart bra cup and light receivers can be on the opposite half of the cup. In an example, light emitters can be on a first side of an oblique virtual plane through a smart bra cup and light receivers can be on a second side (e.g. the opposite side) of the oblique virtual plane, wherein light from the light emitters is transmitted through breast tissue before being received by the light receivers. In another example, a smart bra cup can include a concave array of light emitters and receivers. In an example, light emitters (e.g. LEDs) and light receivers (e.g. photodetectors) in a cup of smart bra can be configured in a plurality of radial spokes of light emitters and receivers extending out from the center (and/or ventral apex) of the cup. In another example, light emitters and receivers in a smart bra cup can be configured in a spiral (e.g. from the base of the cup to the center and/or apex of the cup). In one embodiment, light emitters and/or receivers in a smart bra cup can be configured in a hexagonal (e.g. honeycomb) grid. In another example, light emitters and/or receivers in a smart bra cup can be configured in an orthogonal grid. In an example, light emitters in a smart bra cup can be configured in a spiral (e.g. from the base of the cup to the center and/or apex of the cup).

In an example, a smart bra cup can comprise a beehive configuration with three rings of light emitters and/or detectors around a person's breast. In an example, a smart bra cup can comprise a stack of multiple rings of light emitters and/or detectors around a person's breast. In an example, a smart bra cup can comprise an alternating sequence of light emitters and light receivers in a (vertical) ring around a breast. In an example, a smart bra cup can comprising a plurality of optical sensor rings, wherein each ring further comprises a repeating sequence of light emitters and light receivers. In an example, light emitters (e.g. LEDs) and light receivers (e.g. photodetectors) in a cup of smart bra can be configured in a dorsal-to-ventral sequence of rings of light emitters and receivers on the cup. In another example, light emitters (e.g. LEDs) in a cup of smart bra can be configured in a dorsal-to-ventral sequence of rings of light emitters on the cup. In an example, there can be rings of optical sensors around the inner circumference of a proximal inner ring, wherein each ring further comprises an alternating sequence of light emitters and light receivers.

In an example, distances between light emitters and light receivers on a smart bra cup can be less for portions of the cup which are closer to the center of the cup than for portions of the cup which are farther from the center of the cup. In an example, the density of light emitters and/or light receivers can be greater in selected quadrants (e.g. the upper-outer quadrant) of a smart bra cup than in other quadrants of the cup. In another example, the density of light emitters in the upper-outer quadrant of a smart bra cup can be greater than in the lower-inner quadrant of the cup. In one embodiment, the density of light receivers in a smart bra cup can be greater for portions of the cup which farther from the chest wall and less for portions of the cup which are closer to the chest wall. In an example, the density of optical sensors (e.g. light emitters and/or light receivers) in a smart bra cup can be greater in the lower half of the cup than in the upper half of the cup. In an example, the density of optical sensors in a smart bra cup can be greater for portions of the cup which farther from the chest wall and less for portions of the cup which are closer to the chest wall.

In an example, a smart bra can comprise a plurality of light emitters which emit light at different angles and/or vectors. In an example, the vectors along which light beams from light emitters are transmitted through breast tissue can vary with distance of light emitters from the apex and/or center of a smart bra cup. In another example, a light emitter in a smart bra scan emit light at different wavelengths at different times, ranging from red light (e.g. approximately 660 nm) to near infrared light (e.g. approximately 940 nm). In an example, a smart bra can comprise a light emitter which emits light at different wavelengths at different times. In another example, different light emitters on a smart bra cup can be activated at different times to create different light transmission vectors between light emitters and light receivers at different times. In an example, LEDs emitting red light and LEDs emitting near infrared light in a smart bra can be flashed, pulsed, and/or activated in an alternating and repeating (e.g. red, near infrared, red, near infrared, etc.) sequence.

In an example, the vector long which a light emitter on a smart bra cup emits light into a breast can be moved, adjusted, and/or controlled by changing an electrical field which moves the light emitter. In one embodiment, a smart bra can comprise a plurality of electromagnetic actuators which move light emitters in different directions or to different locations on a cup. In an example, a smart bra cup can comprise a plurality of optical modules, wherein each optical module includes a light emitter, a movable light guide, and a light receiver. In one embodiment, changing the power and/or polarity of electrical energy transmitted through electrodes on a smart bra cup can change an electromagnetic field which moves (e.g. pivots or rotates) a light guide on the cup. In an example, the vectors along which light beams from light emitters are transmitted through breast tissue can be changed by moving a micromirror array which reflects these light beams.

In an example, a smart bra cup can comprise a plurality of optical sensor modules, wherein each module further comprises a near infrared light emitter, a red light emitter, a green light emitter, and at least one light receiver. In another example, a smart bra cup can comprise a plurality of optical sensor modules, wherein each module further comprises at least one light emitter and at least one light receiver. In an example, a light receiver can be a light-sensing detector. In another example, a smart bra can include polymer optical fibers which act as light emitters.

In an example, a smart bra cup can comprise upper and lower expandable chambers (e.g. above and below the breast) which gently compress a breast from having a first (vertical-plane) cross-sectional shape to a having a second (vertical-plane) cross-sectional shape, wherein the second shape has a shorter vertical dimension and/or a thinner vertical width than the first shape. In another example, a smart bra cup can gently compress a person's breast by controlled expansion of a plurality of expandable (e.g. inflatable) chambers. In an example, an expandable chamber can be an inflatable chamber (e.g. balloon). In an example, an expandable chamber can be a fluid-filled chamber.

In an example, a smart bra can include a compartment on the back strap; wherein the compartment further comprises a battery, data processor, and pump; and wherein the pump fills one or more expandable chambers in a smart bra cup with a flowable substance (e.g. gas, liquid, or gel). In an example, a smart bra can include a manual pump which is operated by the person wearing the bra, wherein the pump sends a flowable substance (e.g. air or saline) into expandable chambers in a smart bra cup, and wherein the pump is integrated into the back strap of the bra. In an example, a smart bra can include a plurality of tubes which conduct a flowable substance from a pump to a plurality of expandable chambers in a cup, wherein there is a separate tube for each chamber. In another example, the flow rate of a flowable substance which is pumped into an expandable chamber in a smart bra cup can decrease with time after initial activation of a pump.

In an example, a smart bra can include a comfort-control safety mechanism which controls expansion of one or more expandable chambers in a smart bra, wherein the control mechanism requires active and/or continued pressure (e.g. button pressing) on the mechanism by the person wearing the bra in order to continue expansion of the chambers, and wherein expansion of the chambers stops (and/or even reverses) when the person stops exerting pressure on the mechanism (e.g. stops pressing the button).

In an example, a smart bra cup can comprise a plurality of expandable chambers, wherein each chamber has a separate tube in communication with its interior, and wherein this enables selective, differential, and independent expansion of individual expandable chambers. In another example, a smart bra cup can comprise an array of individually-controllable expandable chambers, wherein selective and differential expansion of chambers in this array enable customizing the shape of the cup to the size and shape of a specific person's breast. In one embodiment, a smart bra can comprise a plurality of expandable chambers which are expanded by different amounts, wherein chambers that are closer to the bra cup center are expanded less than chambers which are farther from the bra cup center. In an example, an expandable chamber that is between two other expandable chambers can be expanded more than the other two chambers. In another example, the flow rate of a flowable substance into an expandable chamber in a smart bra cup can vary over time.

In an example, a smart bra can comprise a plurality of expandable chambers which are expanded at different times, wherein chambers that are closer to the bra cup center are expanded before chambers which are farther from the bra cup center. In an example, expandable chambers in a sequence of expandable chambers in a smart bra cup can be sequentially-expanded at different times to create a peristaltic wave which compresses a person's breast in the cup.

In an example, a smart bra cup can comprise a first concave expandable chamber on a lower-inner half of the cup and a second concave expandable chamber on an upper-outer half of the cup. In another example, a smart bra cup can comprise a first expandable chamber on a lower-inner half of the cup and a second expandable chamber on an upper-outer half of the cup. In an example, a smart bra cup can include a concave-shaped expandable chamber (e.g. a chamber which is expanded by being filled with a flowable substance) in the lower half of the cup. In an example, a smart bra cup with expandable chambers and optical sensors (e.g. light emitters and detectors) on opposite sides of an oblique plane can be advantageous for better optical scanning and/or imaging of the upper-outer quadrant of the breast and the Tail of Spence, which are areas where abnormal tissue (e.g. tumors) are more likely to develop.

In an example, a first expandable chamber in a smart bra cup which is closer to the center of the cup can be smaller than a second expandable chamber in the smart bra cup which is farther from the center of the cup. In an example, a smart bra cup can comprise a first expandable chamber on a first side of the cup and a second expandable chamber on a second side of the cup, wherein the second side is opposite the first side, and wherein the second expandable chamber expands to a size which is at least 50% larger than that of the first expandable chamber. In an example, an expandable chamber that is between two other expandable chambers can be larger than the other two chambers. In one embodiment, a lower half of a smart bra cup can have more expandable chambers than an upper half of the cup.

In an example, a smart bra cup can comprise a plurality of keystone and/or trapezoid shaped expandable chambers along the lower half of the cup when worn. In an example, a smart bra cup can comprise a plurality of longitudinal (e.g. cylindrical) expandable (e.g. inflatable) chambers which gently compress a breast within the cup when the chambers are expanded. In another example, a smart bra cup can comprise a toroidal array of expandable chambers which encircle the base on a person's breast. In an example, a smart bra cup can comprise an annular array (e.g. ring) of expandable chambers which encircle the base on a person's breast on the chest wall. In another example, an expandable chamber can be shaped like a section of a circle, ring, or torus. In an example, an expandable chamber in a smart bra cup can be radially asymmetric. In an example, an expandable chamber in a smart bra cup can have an annular section (e.g. half) shape.

In an example, a (crescent-shaped or elliptical) expandable chamber in a smart bra cup can have a longitudinal axis which intersects a line parallel to the bustline vector of the bra at an angle between 20 and 45 degrees, wherein the bustline vector is defined as a horizontal line that connects the apexes of the right and left cups of the bra. In an example, a (crescent-shaped or elliptical) expandable chamber in a smart bra cup can have a longitudinal axis which is parallel to the bustline vector of the bra, wherein the bustline vector is defined as a horizontal line that connects the apexes of the right and left cups of the bra. In an example, a smart bra cup can comprise a first crescent-shaped expandable chamber on the lower half of the cup and a second crescent-shaped expandable chamber on the upper half of the cup.

In an example, a smart bra cup can comprise: a (frustum shaped) rigid outer ring around a person's breast close to the chest wall; a concave array of optical components (light emitters and/or light receivers) around the person's breast; and two or more crescent-shaped expandable chambers between the rigid outer ring and the concave array of optical components, wherein the expandable chambers are expanded by being filled with a flowable substance (e.g. a gas, fluid, or gel), wherein a first crescent-shaped expandable chamber is on a first half of the cup, and wherein a second crescent-shape expandable chamber is on a second half of the cup which is opposite the first half.

In an example, a smart bra cup can comprise: a frustum-shaped rigid outer ring around a person's breast close to the chest wall; a concave array of light emitters and/or light receivers around the person's breast; and two or more crescent-shaped expandable chambers between the rigid outer ring and the concave array, wherein the expandable chambers are expanded by being filled with a flowable substance (e.g. a gas, fluid, or gel), wherein a first crescent-shaped expandable chamber is on the right half of the cup, and wherein a second crescent-shape expandable chamber is on the left half of the cup.

In an example, a smart bra cup can include an expandable chamber (e.g. a chamber which is expanded by being filled with a flowable substance) in the lower half of the cup, wherein the cross-section of the expandable chamber has a crescent shape. In an example, an expandable chamber in a smart bra cup can have a crescent shape. In another example, multiple expandable chambers in a smart bra cup can collectively form a crescent, partial moon, and/or banana shape. In an example, a smart bra can comprise a plurality of toroidal expandable chambers and a plurality of light emitters, wherein the light emitters are located in the central openings of the toroidal expandable chambers.

In an example, a proximal inner ring in a smart bra cup can encircle a person's breast adjacent to and/or in contact with the person's chest wall. In an example, a proximal outer ring of a smart bra cup can have a frustal, cylindrical, annular, or toroidal shape. In an example, a smart bra cup can comprise: (a) an outer frustum-shaped layer around a person's breast, wherein the outer layer is a first average distance from the person's breast; (b) an inner frustum-shaped layer around the person's breast, wherein the inner layer is a second average distance from the person's breast, wherein the second average distance is less than the first average distance; (c) a plurality of expandable components between the inner layer and the outer layer; and (d) a plurality of optical components (light emitters and/or light receivers) around the person's breast between the inner layer and the person's breast. In an example, the longitudinal axis of a proximal inner ring in a smart bra cup can be equal to the longitudinal axis of a proximal outer ring, but the lateral axis (e.g. perpendicular to the longitudinal axis) of the proximal inner ring can be less than the lateral axis (e.g. perpendicular to the longitudinal axis) of the proximal outer ring. In one embodiment, a first portion of an expandable chamber in a smart bra cup which is closer to the surface of a person's breast can be wider than a second portion of the chamber in the smart bra cup which is farther from the person's breast.

In an example, a smart bra cup can comprise: a first arcuate section (or layer) which is configured to be a first distance from the surface of a person's breast; a second arcuate section (or layer) which is configured to be a second distance from the surface of the person's breast, wherein the second distance is greater than the first distance; one or more elastic bands (or strips) connecting the first arcuate section to the second arcuate section; and two or more expandable chambers between the first arcuate section (or layer) and the second arcuate section (or layer), wherein expansion of the expandable chambers gently compresses the breast by pushing the first arcuate section (or layer) toward the breast, and wherein the one or more elastic bands are between the expandable chambers.

In an example, a smart bra cup can comprise: a first arcuate section (or layer) which is configured to be a first distance from the surface of a person's breast; a second arcuate section (or layer) which is configured to be a second distance from the surface of the person's breast, wherein the second distance is greater than the first distance; a plurality of expandable chambers between the first arcuate section (or layer) and the second arcuate section (or layer), wherein expansion of the expandable chambers gently compresses the breast by pushing the first arcuate section (or layer) toward the breast; and a plurality of elastic bands (or strips) connecting the first arcuate section (or layer) and the second arcuate section (or layer), wherein the elastic bands (or strips) are between expandable chambers.

In an example, a smart bra cup can comprise: a first portion on a first half of the cup; and a second portion on a second half of the cup; wherein the first portion further comprises a first rigid outer layer, a first expandable chamber layer, and a first inner optical layer (with light emitters, light receivers, or both), wherein the inner layer is closest to a breast when the cup is worn; and wherein the second portion further comprises a second rigid outer layer, a second expandable chamber layer, and a second inner optical layer (with light emitters, light receivers, or both), wherein the inner layer is closest to a breast when the cup is worn.

In an example, a smart bra cup can comprise a deformable gel layer between optical components (e.g. light emitters and/or receivers) and the surface of a person's breast. In an example, a smart bra cup can have four layers: a rigid outer layer (e.g. made from a rigid polymer or reinforced with metal wires); an expandable layer (e.g. a chamber which is expanded by being filled with a gas, liquid, or gel); an optical layer (e.g. a layer with a plurality of light emitters and receivers); and a inner compliant layer (e.g. made with an elastomeric polymer). In another example, a smart bra cup can include an inner layer of opaque elastomeric polymer (e.g. PDMS which has been dyed black), wherein there is an array of recesses in this inner layer, and wherein there are optical elements (e.g. light emitters and/or receivers) in the recesses.

In an example, the inner-most layer of a smart bra cup can be between 2 mm and 6 mm thick. In another example, the inner-most layer of a smart bra cup can be made from polydimethylsiloxane (PDMS). In an example, the inner-most layer of a smart bra cup can be transparent. In an example, the innermost layer of a smart bra cup can comprise a 3 mm to 9 mm elastomeric polymer layer with openings (e.g. holes) for light emitters and light receivers. In an example, the innermost layer of a smart bra cup can comprise an elastomeric polymer layer (e.g. PDMS) with openings (e.g. holes) for light emitters and light receivers. In an example, the innermost layer of a smart bra cup can comprise an opaque elastomeric polymer (e.g. PDMS which is dyed black). In one embodiment, the innermost layer of a smart bra cup can comprise an opaque elastomeric polymer. In an example, the inner-most layer of a smart bra cup can have a Young's modulus between 0.50 and 4.00. In another example, the inner-most layer of a smart bra cup can have one or more optical parameters (e.g. optical absorption coefficient, optical scattering coefficient, and/or anisotropy factor) with values which are within plus or minus 20% of the corresponding mean values for normal breast tissue.

In an example, a distal concave portion (e.g. distal dome) of a smart bra cup can be thinner than other portions of the cup. In another example, a distal concave portion of a smart bra cup can span between 30% and 70% of the dorsal-to-ventral depth of the cup. In an example, the elasticity and/or flexibility of an distal concave portion of a smart bra cup can allow a person's breast to extend in a ventral (e.g. frontal) direction when the base of the breast is compressed by expansion of expandable chambers.

In an example, a smart bra cup can comprise outer-side and inner-side expandable chambers (e.g. to the right and left of the breast) which gently compress a breast from having a first (horizontal-plane) cross-sectional shape to a having a second (horizontal-plane) cross-sectional shape, wherein the second shape has a shorter horizontal dimension and/or a thinner horizontal width than the first shape. In another example, the longitudinal axis of a proximal inner ring can intersect a line which is parallel to a bustline vector at an angle between 30 and 60 degrees.

In an example, a smart bra can include electroconductive pathways which are created by embroidering conductive and elastic material on the fabric of the bra. In an example, a smart bra can include electroconductive pathways which are created by printing conductive ink onto the fabric of the bra. In an example, a smart bra can include undulating (e.g. sinusoidal, serpentine or zigzag) electroconductive thread which transmits electricity to light emitters (e.g. LEDs) on a bra cup. In an example, a smart bra can include undulating (e.g. sinusoidal, serpentine or zigzag) insulated wires which transmit electricity to light emitters (e.g. LEDs). In an example, a smart bra cup can comprise one or more elastic electroconductive pathways which are configured in a nested (e.g. concentric) rings.

In an example, a smart bra cup can comprise one or more elastic electroconductive pathways which are configured in a stacked coaxial rings. In one embodiment, a smart bra cup can include a plurality of elastic electroconductive pathways which provide electromagnetic communication among light emitters, light receivers, and a data processor on the bra. In another example, electroconductive pathways can be created on a smart bra by printing silver and/or carbon based ink onto the fabric of the bra in an undulating (e.g. sinusoidal, serpentine or zigzag) pattern. In an example, electroconductive pathways on a smart bra can be formed by printing ink onto the fabric of the bra, wherein the ink is a mixture of conductive and insulating materials.

In an example, a pressure sensor can be in fluid communication with the interior of an expandable chamber. In an example, a smart bra can comprise a plurality of expandable chambers which are expanded to different interior pressure levels, wherein chambers that are closer to the bra cup center are expanded to lower pressure levels than chambers which are farther from the bra cup center. In another example, a smart bra can include a compression-monitoring sensor which monitors pressure exerted by the interior of a cup on a person's breast. In an example, a smart bra with a plurality of expandable chambers can include a plurality of pressure sensors which are in fluid communication with the interiors of the expandable chambers. In an example, the flow rate of a flowable substance which is pumped into an expandable chamber in a smart bra cup can decrease as the internal pressure of the expandable chamber increases. In an example, the flow rate of a flowable substance which is pumped into an expandable chamber in a smart bra cup can decrease as the pressure within the expandable chamber approaches a selected (e.g. target) pressure level. In another example, a pressure sensor can measure the pressure between a smart bra cup and a person's breast.

In an example, a smart bra cup can comprise on or more piezoelectric bands which contract when electrical energy is applied to them. In an example, removable adhesive strips can be attached to the frame of a smart bra to enable optical scanning closer to a chest wall. In one embodiment, the frame of a smart bra cup can be gently and temporarily adhered to a person's chest wall. In an example, a wearable device for optical scanning of a person's breast to detect abnormal tissue can comprise: a convex (e.g. circular or elliptical) transparent tube which is placed on the chest wall around the person's breast; a horizontal arcuate (e.g. semicircular) transparent tube which spans from one side of the convex transparent tube to the other; and a vertical arcuate (e.g. semicircular) transparent tube which spans from one side of the convex transparent tube to the other; wherein each of the tubes contains a plurality of light emitters (e.g. LEDs) and light receivers (e.g. photodetectors).

In an example, a bra insert with optical sensors can be rotated between multiple insertions and removals to scan a breast from different orientations, angles, and/or locations. In an example, a smart bra cup can have a pocket, pouch, and/or compartment into which an optical sensor module is be removably inserted. In another example, a smart bra system can comprise a smart bra cup, a cup insert with optical sensors and electronic components, and a plurality of connection mechanisms (e.g. snaps, clips, plugs, hook-and-eye material, or magnets) which removably-attach the cup insert to the smart bra cup at selected locations, wherein this system enables the optical sensors and electronic components to be removed from the smart bra before the bra is washing, while also ensuring that the bra insert is positioned in the same location on a breast for scans at different times for more-precise longitudinal analysis of changes in breast tissue for detection of abnormal breast tissue. In an example, optical and/or data processing modules in a smart bra can be removed before the smart bra is washed. In another example, this device can be embodied in a smart bra cup insert which is removably-inserted between a cup of a conventional bra and a person's breast.

In an example, a wearable device with optical sensors for detecting abnormal breast tissue can be embodied in a circular adhesive patch, stick, or bandage which encircles a person's breast. In an example, a wearable device with optical sensors for detecting abnormal breast tissue can be embodied in a concave elastomeric adhesive patch, stick, or bandage which is place on a person's breast. In an example, a wearable device with optical sensors for detecting abnormal breast tissue can be embodied in an annular elastomeric adhesive patch, stick, or bandage which encircles a person's breast. In an example, a smart bra can further comprise a power source (e.g. battery). In an example, a smart bra can further comprise one or more additional components selected from the group consisting of: a pump, a power source (e.g. battery), electroconductive pathways which transmit electrical energy to the light emitters, a data processor, a data transmitter, a data receiver, a pressure sensor, a motion sensor, a piezoelectric actuators, and an electromagnetic actuator.

In an example, a smart bra with optical sensors can be used to periodically (e.g. daily, weekly, monthly, or annually) scan a person's breast to measure changes over time in the molecular composition of the breast. In an example, the effect of transmission through breast tissue on the amplitude and/or spectrum of light can be analyzed longitudinally (e.g. at a number of different times) in order to identify (the development or growth of) abnormal breast tissue.

In an example, analysis of how light from light emitters in a smart bra is changed by transmission through breast tissue can be used to measure levels and/or concentration locations of oxygenated hemoglobin in breast tissue. In an example, analysis of how red and near infrared light from light emitters in a smart bra is changed by transmission through breast tissue can be used to measure levels and/or concentration locations of deoxygenated hemoglobin in breast tissue. In another example, changes in the direction of light caused by its transmission through (or reflection from) breast tissue can be analyzed to identify the levels, concentrations, and/or locations of specific biological substances (e.g. markers associated with abnormal tissue) in the breast tissue. In an example, light which has been transmitted through and/or reflected from breast tissue can be analyzed to detect and/or image levels, concentrations, and/or locations of deoxyhemoglobin in breast tissue. In another example, light which has been transmitted through and/or reflected from breast tissue can be analyzed to detect and/or image levels, concentrations, and/or locations of oxygen in breast tissue. In an example, light which has been transmitted through and/or reflected from breast tissue can be analyzed to detect and/or image the locations and configurations of lymphatics in breast tissue.

In an example, changes in the amplitude and/or spectral distribution of light from light emitters on a smart bra which are caused by its transmission through breast tissue can be analyzed to create a three-dimensional image of the breast tissue. In an example, data from a smart bra concerning transmission of light beams through breast tissue along multiple vectors in multiple cross-sections of the breast can be compiled to create a three-dimensional model (e.g. three-dimensional image) of the breast.

In an example, a smart bra can be used to analyze changes in breast tissue composition over time which could indicate abnormal breast tissue growth. In another example, changes in the effect of transmission through breast tissue on the amplitude and/or spectrum of light from a first time to a second time can be analyzed to identify abnormal breast tissue. In an example, data from a smart bra concerning transmission of light beams through breast tissue along multiple vectors in multiple cross-sections of the breast can be analyzed to image, detect, locate, and/or characterize abnormal tissue in the breast. In another example, light which has been transmitted from a light emitter to a light receiver along different vectors through breast tissue can be analyzed to identify the presence, composition, shape, size, and/or location of abnormal breast tissue. In an example, the amplitude, intensity, and/or brightness of light from a light emitter in a smart bra can be automatically increased or decreased based on analysis of light received from a light receiver in the smart bra.

In an example, light which has been transmitted from a light emitter to a light receiver through breast tissue can be analyzed using Diffuse Correlation Spectroscopy (DCS). In one embodiment, light which has been transmitted from a light emitter to a light receiver through breast tissue can be analyzed using functional Near-Infrared Spectroscopy (fNIRS). In an example, light which has been transmitted from a light emitter to a light receiver through breast tissue can be analyzed using Time Reversal Optical Tomography (TROT).

In an example, a smart bra can be made with a combination of elastane and polyester. In an example, a rigid frame for a cup in an optical smart bra can comprise a metal wire which is enclosed in fabric. In another example, the frame for a cup in an optical smart bra can comprise four segments: a lower upward-opening concave partial-annular segment; an upper downward-opening concave segment; and two elastic segments which connect the ends of the lower and upper partial-annular segments. In an example, the frame for a cup in an optical smart bra can comprise two partial-annular segments. In another example, a proximal inner ring in a smart bra cup can be more flexible, more elastic, and/or less rigid than a proximal outer ring in the cup so that expansion of expandable chambers moves the inner ring more than it moves the outer ring. In one embodiment, portions of a smart bra cup which are farther from a chest wall can be more elastic, more flexible, and/or less rigid than other areas of the cup. In another example, the proximal (e.g. closer to chest wall) portion of a smart bra cup can be less elastic, less flexible, and/or more rigid than the distal (e.g. farther from the chest wall) portion of the cup.

In an example, a smart bra cup can comprise: a lower crescent-shaped portion on a lower half of the cup; and a upper crescent-shaped portion on a upper half of the cup; wherein the lower portion further comprises a lower rigid outer layer, a lower expandable chamber layer, and a lower inner optical layer (with light emitters, light receivers, or both), wherein the inner layer is closest to a breast when the cup is worn; and wherein the upper portion further comprises a upper rigid outer layer, a upper expandable chamber layer, and a upper inner optical layer (with light emitters, light receivers, or both), wherein the inner layer is closest to a breast when the cup is worn. In an example, a smart bra cup can have a tear-drop shape (e.g. outer-perimeter or frame shape), wherein the apex of the tear drop covers the Tail of Spence. In an example, a smart bra frame can encompass a Tail of Spence. In an example, smart bra can include an arm sleeve portion for better coverage of the Tail of Spence. In an example, the outer surface of a smart bra cup can have a hemi-ellipsoidal shape.

In an example, light emitters in a smart bra can be VCSELs (vertical-Cavity Surface-Emitting Lasers). In an example, light emitters in a smart bra can emit short-wave infrared light (e.g. over 1000 nm). In another example, light emitters in a smart bra can include near-infrared (NIR) light emitters (e.g. LEDs) which emit light in range of 910 nm to 970 nm. In an example, light emitters in a smart bra can be connected to fabric by high-frequency vibrations. In another example, light emitters in a smart bra can be connected to fabric by ultrasonic bonding. In an example, light emitters in a smart bra can be connected via copper-coated polyester thread or yarn.

In an example, a light emitter (e.g. LED) can be encapsulated by placing the emitter in a mold and pouring a liquid transparent polymer (e.g. PDMS) over it. In an example, a light emitter in a smart bra can be encapsulated in polyurethane to protect against damage when the bra is washed. In one embodiment, a smart bra can comprise light emitters, light receivers, and electroconductive pathways which are encapsulated in an elastomeric polymer. In an example, light emitters (e.g. LEDs) in smart bra can be embedded in elastomeric polymer material (e.g. PDMS). In another example, optical fibers in fabric used to make a smart bra can be embedded in elastomeric polymer material (e.g. PDMS).

In an example, light emitters can be on a first side of a smart bra cup and light receivers can be on a second side (e.g. the opposite side) of the cup, wherein light from the light emitters is transmitted through breast tissue before being received by the light receivers. In another example, light emitters can be on a lower side of a smart bra cup and light receivers can be on an upper side of the cup, wherein light from the light emitters is transmitted through breast tissue before being received by the light receivers.

In an example, a smart bra cup can include a concave array of light emitters. In another example, light emitters (e.g. LEDs) in a cup of smart bra can be configured in a plurality of radial spokes of light emitters extending out from the center (and/or ventral apex) of the cup. In an example, light emitters and/or light receivers in a smart bra cup can be arranged along radial spokes. In an example, light emitters and/or receivers in a smart bra cup can be configured in a hub-and-spoke array. In an example, light emitters and/or receivers in a smart bra cup can be configured in undulating rings around a circumference of the cup.

In an example, a smart bra cup can comprise a beehive configuration of rings of light emitters and/or detectors around a person's breast. In one embodiment, a smart bra cup can comprise a plurality of alternating (vertical) rings of light emitters and light receivers around a breast. In an example, a smart bra cup can comprise a stack of multiple rings of light emitters and/or detectors around a person's breast. In another example, a smart bra cup can comprise four nested (e.g. concentric) rings of light emitters and/or detectors around a person's breast. In one embodiment, a smart bra cup can comprising a repeating sequence of light emitters and light receivers around its perimeter. In another example, light emitters (e.g. LEDs) and light receivers (e.g. photodetectors) in a cup of smart bra can be configured in a plurality of nested (e.g. concentric) rings of light emitters and receivers around the center (and/or ventral apex) of the cup. In an example, light emitters (e.g. LEDs) in a cup of smart bra can be configured in a plurality of nested (e.g. concentric) rings of light emitters around the center (and/or ventral apex) of the cup.

In an example, distances between light emitters and light receivers on a smart bra cup can be greater for portions of the cup which are closer to the center of the cup than for portions of the cup which are farther from the center of the cup. In an example, distances between light emitters and light receivers on a smart bra cup can be less for proximal (e.g. closer to the chest wall) portions of the cup than for distal (e.g. farther from the chest wall) portions of the cup. In an example, the density of light emitters in a smart bra cup can be greater for portions of the cup which closer to the chest wall and less for portions of the cup which are farther from the chest wall.

In an example, the density of light emitters in the upper-outer quadrant of a smart bra cup can be less than in the lower-inner quadrant of the cup. In an example, the density of light receivers in the upper-outer quadrant of a smart bra cup can be greater than in the lower-inner quadrant of the cup. In an example, the density of optical sensors (e.g. light emitters and/or light receivers) in a smart bra cup can be greater in the upper half of the cup than in the lower half of the cup. In one embodiment, the density of optical sensors in the upper-outer quadrant of a smart bra cup can be greater than in the lower-inner quadrant of the cup. In another example, the angles between the surface of a smart bra cup and light beams emitted from light emitters toward the surface of a breast can decrease with the distance of the light emitters from the apex of the cup.

In an example, a light emitter in a smart bra scan emit light at different wavelengths at different times, ranging from green light (e.g. approximately 530 nm) to near infrared light (e.g. approximately 940 nm). In another example, a light emitter in a smart bra scan emit light at different wavelengths at different times. In an example, a smart bra can comprise a plurality of expandable chambers which are expanded at different times. In another example, different light emitters on a smart bra cup with different colors and/or spectral ranges can be activated at different times. In an example, a smart bra cup can comprise: a convex (e.g. circular or elliptical) transparent tube which encircles a person's breast; one or more light emitters and/or light receivers inside the tube which move around (a portion of) the circumference of the tube to scan breast tissue from different locations and/or angles.

In an example, a smart bra can comprise a plurality of actuators which move light emitters in different directions or to different locations on a cup. In an example, a smart bra can comprise a plurality of electromagnetic actuators which move light receivers in different directions or to different locations on a cup. In an example, a smart bra cup can comprise a plurality of optical sensor modules, wherein each module further comprises at least one light emitter, a light guide (e.g. micromirror), and at least one light receiver. In an example, the vector along which light from a light emitter is transmitted through breast tissue can be changed by moving a light guide (e.g. by pivoting or rotating a micromirror).

In an example, a smart bra cup can comprise a plurality of optical sensor modules, wherein each module further comprises a light emitter and a light receiver. In another example, a smart bra cup can comprise a plurality of optical sensor modules, wherein each module further comprises a near infrared light emitter, a red light emitter, and at least one light receiver. In one embodiment, a smart bra cup can comprise a plurality of optical sensor modules, wherein each module further comprises three light emitters and a light receiver. In another example, a light receiver can be a photodetector. In an example, a smart bra can include polymer optical fibers which are woven as part of the fabric used to make the bra.

In an example, a smart bra cup can gently compress a person's breast by controlled expansion of a plurality of expandable (e.g. inflatable) chambers on the lower half of the cup (when the person's is standing). In an example, an expandable chamber can be a balloon or inflatable bladder. In an example, an expandable chamber can be expanded by a pneumatic mechanism. In an example, an expandable chamber can be expanded by a hydraulic mechanism.

In an example, a smart bra can include a handheld pump which is operated by the person wearing the bra, wherein the pump sends a flowable substance (e.g. air or saline) into expandable chambers in a smart bra cup, and wherein the pump is separate from (but removably-connectable to) the bra. In an example, a smart bra can include a manual pump which is operated by the person wearing the bra, wherein the pump sends a flowable substance (e.g. air or saline) into expandable chambers in a smart bra cup, and wherein the pump is separate from (but removably-connectable to) the bra. In an example, a smart bra can include a plurality of tubes which conduct a flowable substance from a pump to a plurality of expandable chambers in a cup. In another example, a pump can be a separate component which is removably-connected to the smart bra.

In an example, a smart bra can include a control mechanism which controls expansion of one or more expandable chambers in a smart bra, wherein the control mechanism requires active and/or continued pressure (e.g. button pressing) on the mechanism by the person wearing the bra in order to continue expansion of the chambers and wherein expansion of the chambers stops (and/or even reverses) when the person stops exerting pressure on the mechanism (e.g. stops pressing the button). In an example, a smart bra cup can comprise an array of individually-controllable expandable chambers, wherein selective and differential expansion of chambers in this array enable conforming the inner shape of the cup to the size and shape of a specific breast. In another example, expandable chambers can be individually and selectively expanded so that they are configured to compress wider portions of the breast to a greater degree than narrower portions of the breast.

In an example, a smart bra can comprise a plurality of expandable chambers which are expanded by different amounts, wherein chambers that are closer to the bra cup center are expanded more than chambers which are farther from the bra cup center. In another example, expansion of one or more expandable chambers in a smart bra cup can be non-linear, wherein the flow rate of a flowable substance into the expandable chambers decreases with time after initial activation. In an example, the intensity and/or amplitude of light transmitted through a breast can be monitoring as a function of expansion of expandable chambers in a smart bra cup. In an example, expandable chambers in a plurality expandable chambers in a smart bra cup can be expanded at different times. In an example, expandable chambers in a sequence of expandable chambers in a smart bra cup can be sequentially-expanded at different times.

In an example, a smart bra cup can comprise a first concave expandable chamber on the lower half of the cup and a second concave expandable chamber on the upper half of the cup. In an example, a smart bra cup can comprise a first expandable chamber on the lower half of the cup and a second expandable chamber on the upper half of the cup. In an example, a smart bra cup can include a plurality of expandable chambers (e.g. chambers which are expanded by being filled with a flowable substance) in the lower half of the cup, wherein the combined cross-section of the plurality of expandable chambers has a concave shape. In another example, there can be one or more expandable chambers on each side of a smart bra cup in the spaces (e.g. gaps) between a proximal inner ring and a proximal outer ring.

In an example, a lower half of a smart bra cup can have larger expandable chambers than an upper half of the cup. In another example, a smart bra cup can comprise a first expandable chamber on a first side of the cup and a second expandable chamber on a second side of the cup, wherein the second side is opposite the first side, and wherein the second expandable chamber is at least 50% larger than the first expandable chamber. In an example, an expandable chamber which is between two other expandable chambers can be larger than either of the two other expandable chambers.

In an example, a smart bra cup can comprise a beehive configuration of annular (e.g. toroidal) expandable chambers which encircle a breast. In an example, a smart bra cup can comprise a plurality of keystone and/or trapezoid shaped expandable chambers. In an example, a smart bra cup can comprise a stacked array of annular (e.g. toroidal) expandable chambers which encircle a breast. In an example, a smart bra cup can comprise an annular array (e.g. ring) of expandable chambers which encircle the base on a person's breast on the chest wall, wherein the forward-central portion of the cup is highly-elastic to allow the breast to extend forward during expansion. In an example, an expandable chamber can be shaped like a half of a circle, ring, or torus. In an example, an expandable chamber can have a keystone or trapezoidal shape. In another example, an expandable chamber in a smart bra cup can have a concave shape. In one embodiment, an expandable chamber in a smart bra cup can have an torus section (e.g. half) shape.

In an example, a (crescent-shaped or elliptical) expandable chamber in a smart bra cup can have a longitudinal axis which intersects a line parallel to the bustline vector of the bra at an angle between 40 and 50 degrees, wherein the bustline vector is defined as a horizontal line that connects the apexes of the right and left cups of the bra. In an example, a smart bra cup can comprise a first crescent-shaped expandable chamber on a lower-inner half of the cup and a second crescent-shaped expandable chamber on an upper-outer half of the cup, wherein one end of the second crescent-shaped expandable chamber spans the Tail of Spence.

In an example, a smart bra cup can comprise: a (frustum shaped) rigid outer ring around a person's breast close to the chest wall; a concave array of optical components (light emitters and/or light receivers) around the person's breast; and two crescent-shaped expandable chambers between the rigid outer ring and the concave array of optical components, wherein the expandable chambers are expanded by being filled with a flowable substance (e.g. a gas, fluid, or gel), wherein a first crescent-shaped expandable chamber is on a first half of the cup, and wherein a second crescent-shape expandable chamber is on a second half of the cup which is opposite the first half.

In an example, a smart bra cup can comprise: a (frustum shaped) rigid outer ring around a person's breast close to the chest wall; a concave array of optical components (light emitters and/or light receivers) around the person's breast; and two or more crescent-shaped expandable chambers between the rigid outer ring and the concave array of optical components, wherein the expandable chambers are expanded by being filled with a flowable substance (e.g. a gas, fluid, or gel), wherein expansion of the expandable chambers gently compresses the breast, wherein a first crescent-shaped expandable chamber is on a first half of the cup, and wherein a second crescent-shape expandable chamber is on a second half of the cup which is opposite the first half.

In an example, a smart bra cup can include a crescent-shaped expandable chamber (e.g. a chamber which is expanded by being filled with a flowable substance) in the lower half of the cup. In an example, a smart bra cup can include an expandable chamber (e.g. a chamber which is expanded by being filled with a flowable substance) in the lower half of the cup, wherein the cross-section of the expandable chamber has an upwardly-concave crescent shape. In an example, an expandable chamber in a smart bra cup can have a crescent, partial moon, and/or banana shape. In another example, a plurality of expandable chambers on a given side of a smart bra cup can collectively (e.g. together) comprise a crescent or banana shape. In an example, a smart bra cup can include a layer of expandable chambers and optical elements (e.g. light emitters and/or receivers), wherein the optical elements are between the expandable chambers.

In an example, a proximal inner ring of a smart bra cup can have a frustal, toroidal, or annular shape. In another example, a smart bra cup can comprise: (a) a circular outer ring around a person's breast, wherein the outer ring is a first average distance from the person's breast; (b) an oblate-circular, elliptical or oval inner ring around the person's breast, wherein the inner ring is a second average distance from the person's breast, and wherein the second average distance is less than the first average distance; (c) a plurality of expandable components between the inner ring and the outer ring; and (d) a plurality of optical components (light emitters and/or light receivers) around the person's breast between the inner ring and the person's breast.

In an example, a smart bra cup can comprise: (a) an outer ring around a person's breast, wherein the outer ring is a first average distance from the person's breast; (b) an inner ring around the person's breast, wherein the inner ring is a second average distance from the person's breast, wherein the second average distance is less than the first average distance, and wherein the perimeter of the outer ring is closer to being circular than that of the inner ring; (c) a plurality of expandable components between the inner ring and the outer ring; and (d) a plurality of optical components (light emitters and/or light receivers) around the person's breast between the inner ring and the person's breast. In another example, there can be a first difference in length between the longitudinal axis of a proximal inner ring and the longitudinal axis of a proximal outer ring, and a second difference in length between the lateral axis (e.g. perpendicular to the longitudinal axis) of the proximal inner ring and the lateral axis (e.g. perpendicular to the longitudinal axis) of the proximal outer ring, wherein the second difference is greater than the first difference. In an example, a proximal inner ring of a smart bra cup can span between 20% and 70% of the dorsal-to-ventral depth of a smart bra cup.

In an example, a smart bra cup can comprise: a first arcuate section (or layer) which is configured to be a first distance from the surface of a person's breast; a second arcuate section (or layer) which is configured to be a second distance from the surface of the person's breast, wherein the second distance is greater than the first distance; and one or more expandable chambers between the first arcuate section (or layer) and the second arcuate section (or layer), wherein expansion of the one or more expandable chambers gently compresses the breast by pushing the first arcuate section (or layer) toward the breast.

In an example, a smart bra cup can comprise: a first concave section (or layer) which is configured to be a first distance from the surface of a person's breast; a second concave section (or layer) which is configured to be a second distance from the surface of the person's breast, wherein the second distance is greater than the first distance; and one or more expandable chambers between the first concave section (or layer) and the second concave section (or layer), wherein expansion of the one or more expandable chambers gently compresses the breast by pushing the first concave section (or layer) toward the breast.

In an example, a smart bra cup can comprise: a lower-outer portion on a lower-outer half of the cup; and a upper-inner portion on a upper-inner half of the cup; wherein the lower-outer portion further comprises a lower-outer rigid outer layer, a lower-outer expandable chamber layer, and a lower-outer inner optical layer (with light emitters, light receivers, or both), wherein the inner layer is closest to a breast when the cup is worn; and wherein the upper-inner portion further comprises a upper-inner rigid outer layer, a upper-inner expandable chamber layer, and a upper-inner inner optical layer (with light emitters, light receivers, or both), wherein the inner layer is closest to a breast when the cup is worn.

In an example, a smart bra cup can comprise a deformable opaque gel layer between optical components (e.g. light emitters and/or receivers) and the surface of a person's breast. In one embodiment, a smart bra cup can have three layers: a rigid outer layer (e.g. made from a rigid polymer or reinforced with metal wires); an expandable layer (e.g. a chamber which is expanded by being filled with a gas, liquid, or gel); an optical inner layer comprising a layer of opaque elastomeric polymer with holes, openings, and/or recesses and light emitters and receivers in those holes, openings, and/or recesses. In an example, light emitters and/or light receivers in a smart bra can be located between a layer of expandable chambers and an inner layer (e.g. closest to a person's breast) made from opaque elastomeric material (e.g. PDMS).

In an example, the inner-most layer of a smart bra cup can be made from a silicone-based polymer. In an example, the inner-most layer of a smart bra cup can be made from thermoplastic polyurethane. In another example, the innermost layer of a smart bra cup can comprise a 1 mm to 5 mm elastomeric polymer layer with openings (e.g. holes) for light emitters and light receivers. In an example, the innermost layer of a smart bra cup can comprise a 3 mm to 9 mm elastomeric polymer layer. In an example, the innermost layer of a smart bra cup can comprise an elastomeric polymer layer with openings (e.g. holes) for light emitters and light receivers.

In an example, the innermost layer of a smart bra cup can comprise an opaque elastomeric polymer layer (e.g. PDMS which is dyed black) with openings (e.g. holes) for light emitters and light receivers. In an example, the inner-most layer of a smart bra cup can have a Shore durometer level between 5 and 30. In another example, the inner-most layer of a smart bra cup can have an anisotropy factor with a value like that of breast tissue. In an example, the layer of a smart bra cup which is closest to a person's breast can be made with a silicone-based polymer.

In an example, a distal concave portion of a smart bra cup can have a dorsal-to-ventral depth between 1 and 3 inches. In one embodiment, a distal portion of a smart bra cup can be articulated to allow for expansion when a breast is compressed by expansion of expandable chambers in the cup. In an example, the front portion of a smart bra cup can be made with highly-elastic (and/or loose-fitting) fabric which allows a person's breast to extend forward during gentle compression of the breast by expandable chambers.

In an example, a wearable device for optical scanning of a person's breast to detect abnormal tissue can comprise: a convex (e.g. circular or elliptical) transparent tube which is placed on the chest wall around the person's breast; a horizontal arcuate (e.g. semicircular) transparent tube which spans from one side of the convex transparent tube to the other; and a vertical arcuate (e.g. semicircular) transparent tube which spans from one side of the convex transparent tube to the other; wherein each of the tubes contains a plurality of light emitters (e.g. LEDs) and/or light receivers (e.g. photodetectors).

In an example, a smart bra can include a plurality of electroconductive elastomeric polymer (e.g. PDMS which has been doped or impregnated with conductive material) pathways which transmit electrical signals from light receivers (e.g. photodetectors). In an example, a smart bra can include electroconductive pathways which are created by embroidering conductive material on the fabric of the bra. In an example, a smart bra can include undulating (e.g. sinusoidal, serpentine or zigzag) electroconductive thread which transmits electrical signals from light receivers (e.g. photodetectors) on a bra cup.

In an example, a smart bra can include undulating (e.g. sinusoidal, serpentine or zigzag) electroconductive thread which transmits electricity to light emitters (e.g. LEDs) on a bra frame. In another example, a smart bra cup can comprise one or more elastic electroconductive pathways which are configured in a hexagonal mesh (e.g. honeycomb) pattern. In an example, a smart bra cup can comprise one or more elastic electroconductive pathways which are configured in a radial starburst pattern. In another example, a smart bra cup can comprise one or more elastic electroconductive pathways which are configured in an undulating (e.g. sinusoidal, zigzag, or serpentine) pattern. In one embodiment, a smart bra cup can include electroconductive pathways made from a silicone-based elastomeric polymer (e.g. PDMS) which has been doped, impregnated, or coated with conductive material (e.g. silver or carbon particles). In another example, electroconductive pathways can be created on a smart bra by printing silver and/or carbon based ink onto the fabric of the bra.

In an example, a first expandable chamber in a smart bra cup which is closer to the center of the cup can be filled with a flowable substance to a greater interior pressure level than a second expandable chamber in the smart bra cup which is farther from the center of the cup. In an example, a smart bra can automatically shut off a pump when the pressure within an expandable chamber or between a smart bra cup and a person's breast exceeds a selected level. In an example, a smart bra can comprise a plurality of expandable chambers which are expanded to different interior pressure levels. In another example, a smart bra can include a pressure release valve which is in fluid communication with the interior of an expandable chamber in a cup.

In an example, a smart bra with a plurality of expandable chambers can include one or more pressure sensors between the expandable chambers and a person's breast. In an example, the flow rate of a flowable substance which is pumped into an expandable chamber in a smart bra cup can decrease as the pressure level between the cup and a person's breast within the cup approaches a selected (e.g. target) pressure level. In an example, the operation of a pump on a smart bra can be controlled by a pressure sensor between the interior of a cup and a person's breast within the cup. In an example, a smart bra cup can comprise a plurality of piezoelectric rings which gently compress a breast when electrical energy is applied to them. In an example, a smart bra cup can comprise on or more piezoelectric bands which expand when electrical energy is applied to them. In an example, removable adhesive strips can be attached to the frame of a smart bra to reduce movement of a cup while expandable chambers are expanded. In an example, a wearable device for optical scanning of a person's breast to detect abnormal tissue can comprise a convex (e.g. circular or elliptical) transparent tube which is placed on the chest wall around the person's breast, wherein the tube contains a plurality of light emitters (e.g. LEDs) and light receivers (e.g. photodetectors).

In an example, a bra insert with optical modules to detect abnormal breast tissue can be removably-attached to a concave interior of a bra cup by an attachment mechanism (e.g. hook-and-loop material, snap, clip, or magnet) so that it is held in place for optical scanning, but can be removed for washing the smart bra. In an example, a bra insert with optical sensors for detecting abnormal breast tissue can be inserted between the cup of a traditional bra and a person's breast within the cup, wherein the bra insert is attached to the cup by one or more mechanisms selected from the group consisting of: snap, clip, clamp, pin, hook-and-eye material, and magnet. In an example, a smart bra cup can have a pocket, pouch, and/or compartment into which an optical sensor module is inserted to optically scan a person's breast and then removed before the bra is washed.

In an example, a smart bra system can comprise a smart bra cup, a cup insert with optical sensors, and a plurality of connection mechanisms (e.g. snaps, clips, plugs, hook-and-eye material, or magnets) which removably-attach the cup insert to the smart bra cup at selected locations, wherein this system enables the optical sensors to be removed from the smart bra before the bra is washing, while also ensuring that the bra insert is positioned in the same location on a breast for scans at different times for more-precise longitudinal analysis of changes in breast tissue for detection of abnormal breast tissue. In an example, optical and/or electronic components of a smart bra can be modular and/or removable components which can be removed before the bra is washed and replaced after the bra is washed.

In an example, a smart bra can comprise an adhesive ring which is gently adhered to the chest wall where a breast is attached to the chest wall, encompassing the Tail of Spence, the upper outer quadrant, the upper inner quadrant, the lower inner quadrant, and the lower outer quadrant of the breast. In an example, a wearable device with optical sensors for detecting abnormal breast tissue can be embodied in a circular elastomeric adhesive patch, stick, or bandage which encircles a person's breast. In an example, a wearable device with optical sensors for detecting abnormal breast tissue can be embodied in an adhesive patch, stick, or bandage. In an example, a wearable device with optical sensors for detecting abnormal breast tissue can be embodied in an elastomeric adhesive patch, stick, or bandage. In an example, a smart bra can further comprise a wireless data receiver. In an example, a smart bra can include a compartment on the back strap, wherein the compartment further comprises a battery, data processor, and data transmitter.

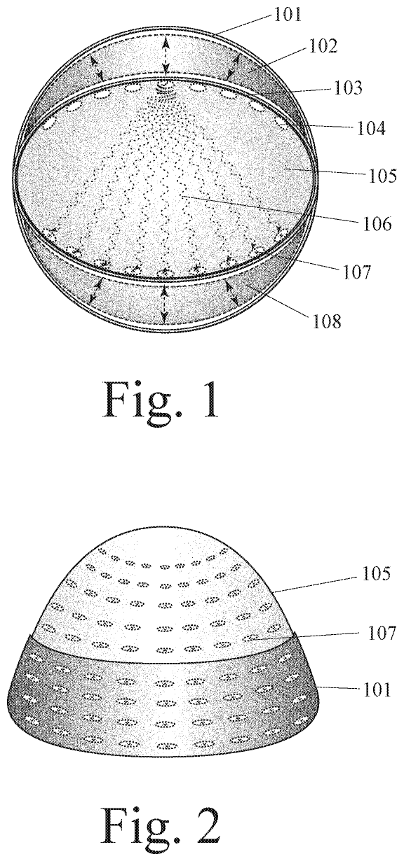

FIGS. 1 and 2 show two views, from two difference perspectives, of an example of a smart bra cup with crescent-shaped expandable chambers and optical sensors on either side of the cup for detecting abnormal breast tissue. FIG. 1 shows a semi-transparent frontal view of this smart bra cup. FIG. 2 shows an upward-facing view of this smart bra cup.

With respect to components, FIGS. 1 and 2 show two views of a smart bra cup comprising: (a) a proximal outer ring 101 which is part of a smart bra cup, wherein the outer ring is configured to encircle a person's breast; (b) a proximal inner ring 103 which is part of the smart bra cup, wherein the inner ring is configured to encircle the person's breast and wherein the inner ring is inside the outer ring; (c) a plurality of expandable chambers (including 102 and 108) between the inner ring and the outer ring; (d) an elastic concave distal portion 105 of the smart bra cup which covers a distal portion of the person's breast; and (e) a plurality of light emitters 104 and light receivers 107 between the inner ring and the person's breast, wherein changes in light 106 emitted from the light emitters and received by the light receivers which are caused by transmission of the light through breast tissue are analyzed to detect, locate, evaluate, characterize, and/or image abnormal breast tissue.

More generally, a smart bra cup can comprise: (a) a proximal outer ring which is part of a smart bra cup, wherein the outer ring is configured to encircle a person's breast; (b) a proximal inner ring which is part of the smart bra cup, wherein the inner ring is configured to encircle the person's breast and wherein the inner ring is inside the outer ring; (c) a plurality of expandable chambers between the inner ring and the outer ring; (d) an elastic concave distal portion of the smart bra cup which covers a distal portion of the person's breast; and (e) a plurality of optical sensors (including light emitters and light receivers) between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

In an example, both the right and left side cups of a smart bra can have optical sensors for detecting abnormal breast tissue. In an example, the bra cup design shown in FIGS. 1 and 2 can be used for one or both cups. In an example, optical sensors can be permanently integrated into a smart bra cup. In another example, a bra cup can have a pocket, pouch, or compartment into which optical sensors (and electronics) can be placed, wherein the optical sensors can be removed before the bra is washed and put back after the bra has been washed. In another example, this device can be embodied in a smart bra cup insert which is removably-inserted between a cup of a conventional bra and a person's breast.

In an example, a smart bra can further comprise a pump and tubes. The pump pumps a flowable substance (e.g. gas, liquid, or gel) through the tubes into expandable chambers. Expansion of the expandable chambers gently compresses a person's breast for more accurate optical scanning over shorter distances. In another example, a pump can be a separate component which is removably-connected to the smart bra. In an example, the person wearing the bra can actively control the operation of a pump so that it can be stopped (or reversed) if compression of the person's breast becomes uncomfortable. In an example, a smart bra can further comprise one or more additional components selected from the group consisting of: a pump, a power source (e.g. battery), electroconductive pathways which transmit electrical energy to the light emitters, a data processor, a data transmitter, a data receiver, a pressure sensor, a motion sensor, a piezoelectric actuators, and an electromagnetic actuator. In an example, one or more of these additional components can be located on a back strap of the smart bra.

In this example, proximal is defined as being closer to a person's chest wall and distal is defined as being farther from the person's chest wall. In an example, a proximal outer ring can encircle a person's breast adjacent to (and/or in contact with) the person's chest wall. In an example, a proximal outer ring can be relatively rigid (e.g. more rigid than the rest of the bra cup). In an example, a proximal outer ring can have a dorsal-to-ventral width between 1 and 3 inches. In an example, a proximal outer ring can span between 20% and 70% of the dorsal-to-ventral depth of a smart bra cup. In an example, a proximal outer ring can have a frustal, cylindrical, annular, or toroidal shape. In an example, the cross-sectional perimeter of a proximal outer ring can have a circular, oblate-circular, elliptical, oval, or teardrop shape.

In an example, a cross-sectional perimeter of a proximal outer ring can have a longitudinal axis. In an example, the longitudinal axis of a proximal outer ring can be parallel to a bustline vector which connects the apexes of right and left side bra cups. In another example, the longitudinal axis of a proximal outer ring can be perpendicular to a line which is parallel to the bustline vector. In another example, the longitudinal axis of a proximal outer ring can intersect a line which is parallel to the bustline vector at an angle between 30 and 60 degrees.

In an example, a proximal inner ring can encircle a person's breast adjacent to and/or in contact with the person's chest wall. In an example, a proximal inner ring can have a frustal shape. In an example, a proximal inner ring can have a cylindrical shape. In an example, a proximal inner ring can be more flexible, more elastic, and/or less rigid than a proximal outer ring so that expansion of expandable chambers moves the inner ring more than it moves the outer ring. In an example, a proximal inner ring can have an annular shape. In an example, a proximal inner ring can have a toroidal shape. In an example, a proximal inner ring can have a circular, oblate-circular, elliptical, or oval cross-sectional perimeter shape. In an example, a proximal inner ring can have a dorsal-to-ventral width between 1 and 3 inches. In an example, a proximal inner ring can span between 20% and 70% of the dorsal-to-ventral depth of a smart bra cup.

In an example, a proximal inner ring can be less circular than a proximal outer ring. In an example, the cross-sectional perimeter of a proximal inner ring can have a longitudinal axis. In an example, the longitudinal axis of a proximal inner ring can be parallel to a bustline vector connecting the apexes of right side and left side bra cups. In another example, the longitudinal axis of a proximal inner ring can be perpendicular to a line which is parallel to the bustline vector. In another example, the longitudinal axis of a proximal inner ring can intersect a line which is parallel to the bustline vector at an angle between 30 and 60 degrees. In an example, there can be two crescent and/or banana shaped spaces (e.g. gaps) between a proximal inner ring and a proximal outer ring (e.g. one on each side of the cup). In an example, a smart bra cup can have two arcuate inner segments (e.g. one arcuate segment on either side of the cup) instead of a continuous inner ring.

In an example, the longitudinal axis of a proximal inner ring can be equal to the longitudinal axis of a proximal outer ring, but the lateral axis (e.g. perpendicular to the longitudinal axis) of the proximal inner ring can be less than the lateral axis (e.g. perpendicular to the longitudinal axis) of the proximal outer ring. In an example, there can be a first difference in length between the longitudinal axis of a proximal inner ring and the longitudinal axis of a proximal outer ring, and a second difference in length between the lateral axis (e.g. perpendicular to the longitudinal axis) of the proximal inner ring and the lateral axis (e.g. perpendicular to the longitudinal axis) of the proximal outer ring, wherein the second difference is greater than the first difference.

In an example, an expandable chamber can be an inflatable chamber (e.g. balloon). In an example, an expandable chamber can be expanded by being filled with a gas (e.g. air). In an example, an expandable chamber can be a fluid-filled chamber. In an example, an expandable chamber can be expanded by being filled with a liquid (e.g. saline solution). In an example, an expandable chamber can have an oblong, crescent, and/or banana shape. In an example, an expandable chamber can have a convex shape. In an example, an expandable chamber can have a keystone, trapezoidal, or annular section shape.

In an example, there can be one or more expandable chambers on each side of a smart bra cup in the spaces (e.g. gaps) between a proximal inner ring and a proximal outer ring. In an example, there can be one expandable chamber on each side of a smart bra cup, one in each of two spaces (e.g. gaps) between a proximal inner ring and a proximal outer ring. In an example, there can be multiple expandable chambers on each side of a smart bra cup. In an example, there can be multiple expandable chambers in each of two spaces (e.g. gaps) between a proximal inner ring and a proximal outer ring.

In an example, there can be two sets of three expandable chambers, wherein there is one set of three expandable chambers on each side (e.g. on opposite sides) of a smart bra cup. In an example, a middle expandable chamber between two other chambers in a set of chambers can be larger than the other two chambers. In an example, a middle expandable chamber between two other chambers in a set of chambers can be expanded more than the other two chambers. In another example, there can be more than three expandable chambers on each side of a smart bra cup. In another example, a plurality of expandable chambers on a given side of a smart bra cup can collectively (e.g. together) comprise a crescent or banana shape.

In an example, a smart bra cup can further comprise a pressure sensor whose data is used to limit (or otherwise control) the expansion of an expandable chamber. In an example, a pressure sensor can be in fluid communication with the interior of an expandable chamber. In another example, a pressure sensor can measure the pressure between a smart bra cup and a person's breast. In an example, a smart bra can automatically shut off a pump when the pressure within an expandable chamber or between a smart bra cup and a person's breast exceeds a selected level.

In an example, a distal concave portion (e.g. distal dome) of a smart bra cup can be more elastic, more flexible, and/or less rigid than other portions of the cup. In an example, a distal concave portion (e.g. distal dome) of a smart bra cup can be thinner than other portions of the cup. In an example, a proximal outer ring can have a first elasticity level, a proximal inner ring can have a second elasticity level, and a distal concave portion can have a third elasticity level, wherein the second level is greater than the first level and the third level is greater than the second level. In an example, a distal concave portion can have a dorsal-to-ventral depth between 1 and 3 inches. In an example, a distal concave portion of a smart bra cup can span between 30% and 70% of the dorsal-to-ventral depth of the cup. In an example, a distal concave portion can have a hemispherical, hemi-ellipsoidal, half oblate-spheroidal, or paraboloidal shape. In an example, the elasticity and/or flexibility of an distal concave portion can allow a person's breast to extend in a ventral (e.g. frontal) direction when the base of the breast is compressed by expansion of expandable chambers.

In an example, light emitters can be on a first side of a smart bra cup and light receivers can be on a second side (e.g. the opposite side) of the cup, wherein light from the light emitters is transmitted through breast tissue before being received by the light receivers. In an example, light emitters can be on a lower side of a smart bra cup and light receivers can be on an upper side of the cup, wherein light from the light emitters is transmitted through breast tissue before being received by the light receivers. In an example, light emitters can be on a right side of a smart bra cup and light receivers can be on a left side of the cup, wherein light from the light emitters is transmitted through breast tissue before being received by the light receivers. In an example, light emitters can be on a first side of an oblique virtual plane through a smart bra cup and light receivers can be on a second side (e.g. the opposite side) of the oblique virtual plane, wherein light from the light emitters is transmitted through breast tissue before being received by the light receivers.

In an example, there can be rings of optical sensors around the inner circumference of a proximal inner ring, wherein each ring further comprises a plurality of light emitters and light receivers. In an example, there can be rings of optical sensors around the inner circumference of a proximal inner ring, wherein each ring further comprises an alternating sequence of light emitters and light receivers. In an example, there can be rings of optical sensors around the circumference of a proximal inner ring, wherein each ring further comprises a sequence of different-color light emitters and light receivers.

In an example, there can be light emitters and light receivers on both a proximal inner ring and on a distal concave portion of a bra cup. In another example, there can be light emitters and light receivers only on the proximal inner ring. In an example, the density of light emitters and/or light receivers can be greater in proximal portions of a smart bra cup than in distal portions of the cup. In an example, the density of light emitters and/or light receivers can be greater in selected quadrants (e.g. the upper-outer quadrant) of a smart bra cup than in other quadrants of the cup. In an example, different light emitters can be activated at different times to create different light transmission vectors between light emitters and light receivers at different times. In an example, light emitters at different radial locations on a smart bra cup can be activated at different times. In an example, different light emitters with different colors and/or spectral ranges can be activated at different times. In an example, the same light emitter can emit light with different colors and/or spectral ranges at different times.

In an example, a smart bra cup can comprise: a proximal outer ring which is part of a smart bra cup, wherein the outer ring is configured to encircle a person's breast; a proximal inner ring which is part of the smart bra cup, wherein the inner ring is configured to encircle the person's breast and wherein the inner ring is inside the outer ring; a plurality of expandable chambers between the inner ring and the outer ring; and a plurality of optical sensors (including light emitters and light receivers) between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

In an example, a smart bra cup can comprise: a proximal outer ring which is part of a smart bra cup, wherein the outer ring is configured to encircle a person's breast; two concave segments on opposite sides of the smart bra cup, wherein the concave segments are inside the outer ring; a plurality of expandable chambers between the concave segments and the outer ring; and a plurality of optical sensors (including light emitters and light receivers) between the concave segments and the person's breast, wherein changes in light emitted from the light emitters and received by the light receivers which are caused by transmission of the light through breast tissue are analyzed to detect, locate, evaluate, characterize, and/or image abnormal breast tissue. Relevant variations discussed elsewhere in this disclosure or in priority-linked disclosures can also be applied to this example.

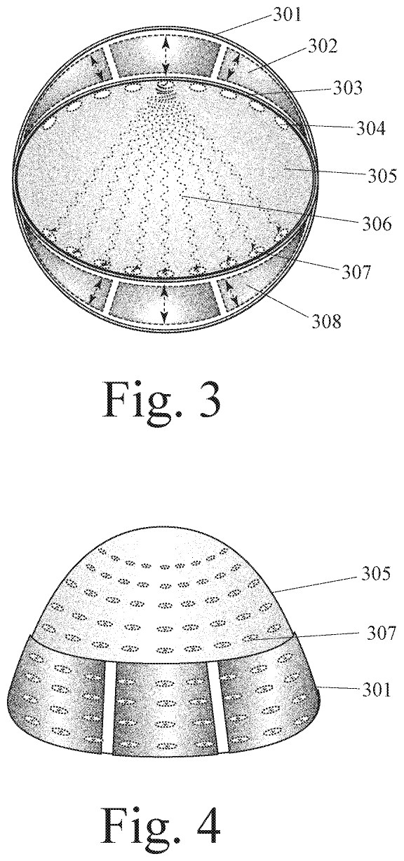

FIGS. 3 and 4 show two views, from two difference perspectives, of an example of a smart bra cup with a plurality of expandable chambers and optical sensors on either side of the cup for detecting abnormal breast tissue. FIG. 3 shows a semi-transparent frontal view of this smart bra cup. FIG. 4 shows an upward-facing view of this smart bra cup.

With respect to components, FIGS. 3 and 4 show two views of a smart bra cup comprising: (a) a proximal outer ring 301 which is part of a smart bra cup, wherein the outer ring is configured to encircle a person's breast; (b) a proximal inner ring 303 which is part of the smart bra cup, wherein the inner ring is configured to encircle the person's breast and wherein the inner ring is inside the outer ring; (c) a plurality of expandable chambers (including 302 and 308) on each side (e.g. on opposite sides) of the cup between the inner ring and the outer ring; (d) an elastic concave distal portion 305 of the smart bra cup which covers a distal portion of the person's breast; and (e) a plurality of light emitters 304 and light receivers 307 between the inner ring and the person's breast, wherein changes in light 306 emitted from the light emitters and received by the light receivers which are caused by transmission of the light through breast tissue are analyzed to detect, locate, evaluate, characterize, and/or image abnormal breast tissue.

More generally, a smart bra cup can comprise: (a) a proximal outer ring which is part of a smart bra cup, wherein the outer ring is configured to encircle a person's breast; (b) a proximal inner ring which is part of the smart bra cup, wherein the inner ring is configured to encircle the person's breast and wherein the inner ring is inside the outer ring; (c) a plurality of expandable chambers on each side (e.g. on opposite sides) of the cup between the inner ring and the outer ring; (d) an elastic concave distal portion of the smart bra cup which covers a distal portion of the person's breast; and (e) a plurality of optical sensors (including light emitters and light receivers) between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

In an example, both the right and left side cups of a smart bra can have optical sensors for detecting abnormal breast tissue. In an example, the bra cup design shown in FIGS. 3 and 4 can be used for one or both cups. In an example, optical sensors can be permanently integrated into a smart bra cup. In another example, a bra cup can have a pocket, pouch, or compartment into which optical sensors (and electronics) can be placed, wherein the optical sensors can be removed before the bra is washed and put back after the bra has been washed. In another example, this device can be embodied in a smart bra cup insert which is removably-inserted between a cup of a conventional bra and a person's breast.

In an example, a smart bra can further comprise a pump and tubes. The pump pumps a flowable substance (e.g. gas, liquid, or gel) through the tubes into expandable chambers. Expansion of the expandable chambers gently compresses a person's breast for more accurate optical scanning over shorter distances. In another example, a pump can be a separate component which is removably-connected to the smart bra. In an example, the person wearing the bra can actively control the operation of a pump so that it can be stopped (or reversed) if compression of the person's breast becomes uncomfortable. In an example, a smart bra can further comprise one or more additional components selected from the group consisting of: a pump, a power source (e.g. battery), electroconductive pathways which transmit electrical energy to the light emitters, a data processor, a data transmitter, a data receiver, a pressure sensor, a motion sensor, a piezoelectric actuators, and an electromagnetic actuator. In an example, one or more of these additional components can be located on a back strap of the smart bra.

In this example, proximal is defined as being closer to a person's chest wall and distal is defined as being farther from the person's chest wall. In an example, a proximal outer ring can encircle a person's breast adjacent to (and/or in contact with) the person's chest wall. In an example, a proximal outer ring can be relatively rigid (e.g. more rigid than the rest of the bra cup). In an example, a proximal outer ring can have a dorsal-to-ventral width between 1 and 3 inches. In an example, a proximal outer ring can span between 20% and 70% of the dorsal-to-ventral depth of a smart bra cup. In an example, a proximal outer ring can have a frustal, cylindrical, annular, or toroidal shape. In an example, the cross-sectional perimeter of a proximal outer ring can have a circular, oblate-circular, elliptical, oval, or teardrop shape.

In an example, a cross-sectional perimeter of a proximal outer ring can have a longitudinal axis. In an example, the longitudinal axis of a proximal outer ring can be parallel to a bustline vector which connects the apexes of right and left side bra cups. In another example, the longitudinal axis of a proximal outer ring can be perpendicular to a line which is parallel to the bustline vector. In another example, the longitudinal axis of a proximal outer ring can intersect a line which is parallel to the bustline vector at an angle between 30 and 60 degrees.

In an example, a proximal inner ring can encircle a person's breast adjacent to and/or in contact with the person's chest wall. In an example, a proximal inner ring can have a frustal shape. In an example, a proximal inner ring can have a cylindrical shape. In an example, a proximal inner ring can be more flexible, more elastic, and/or less rigid than a proximal outer ring so that expansion of expandable chambers moves the inner ring more than it moves the outer ring. In an example, a proximal inner ring can have an annular shape. In an example, a proximal inner ring can have a toroidal shape. In an example, a proximal inner ring can have a circular, oblate-circular, elliptical, or oval cross-sectional perimeter shape. In an example, a proximal inner ring can have a dorsal-to-ventral width between 1 and 3 inches. In an example, a proximal inner ring can span between 20% and 70% of the dorsal-to-ventral depth of a smart bra cup.

In an example, a proximal inner ring can be less circular than a proximal outer ring. In an example, the cross-sectional perimeter of a proximal inner ring can have a longitudinal axis. In an example, the longitudinal axis of a proximal inner ring can be parallel to a bustline vector connecting the apexes of right side and left side bra cups. In another example, the longitudinal axis of a proximal inner ring can be perpendicular to a line which is parallel to the bustline vector. In another example, the longitudinal axis of a proximal inner ring can intersect a line which is parallel to the bustline vector at an angle between 30 and 60 degrees. In an example, there can be two crescent and/or banana shaped spaces (e.g. gaps) between a proximal inner ring and a proximal outer ring (e.g. one on each side of the cup). In an example, a smart bra cup can have two arcuate inner segments (e.g. one arcuate segment on either side of the cup) instead of a continuous inner ring.

In an example, the longitudinal axis of a proximal inner ring can be equal to the longitudinal axis of a proximal outer ring, but the lateral axis (e.g. perpendicular to the longitudinal axis) of the proximal inner ring can be less than the lateral axis (e.g. perpendicular to the longitudinal axis) of the proximal outer ring. In an example, there can be a first difference in length between the longitudinal axis of a proximal inner ring and the longitudinal axis of a proximal outer ring, and a second difference in length between the lateral axis (e.g. perpendicular to the longitudinal axis) of the proximal inner ring and the lateral axis (e.g. perpendicular to the longitudinal axis) of the proximal outer ring, wherein the second difference is greater than the first difference.

In an example, an expandable chamber can be an inflatable chamber (e.g. balloon). In an example, an expandable chamber can be expanded by being filled with a gas (e.g. air). In an example, an expandable chamber can be a fluid-filled chamber. In an example, an expandable chamber can be expanded by being filled with a liquid (e.g. saline solution). In an example, an expandable chamber can have a keystone, trapezoidal, or annular section shape. In this example, there can be multiple expandable chambers on each side of a smart bra cup. In an example, there can be multiple expandable chambers in each of two spaces (e.g. gaps) between a proximal inner ring and a proximal outer ring.

In this example, there are two sets of three expandable chambers, wherein there is one set of three expandable chambers on each side (e.g. on opposite sides) of a smart bra cup. In this example, a middle expandable chamber between two other chambers in a set of chambers is larger than the other two chambers. In an example, a middle expandable chamber between two other chambers in a set of chambers can be expanded more than the other two chambers. In this example, a plurality of expandable chambers on a given side of a smart bra cup can collectively (e.g. together) comprise a crescent or banana shape.

In an example, a smart bra cup can further comprise a pressure sensor whose data is used to limit (or otherwise control) the expansion of an expandable chamber. In an example, a pressure sensor can be in fluid communication with the interior of an expandable chamber. In another example, a pressure sensor can measure the pressure between a smart bra cup and a person's breast. In an example, a smart bra can automatically shut off a pump when the pressure within an expandable chamber or between a smart bra cup and a person's breast exceeds a selected level.

In an example, a distal concave portion (e.g. distal dome) of a smart bra cup can be more elastic, more flexible, and/or less rigid than other portions of the cup. In an example, a distal concave portion (e.g. distal dome) of a smart bra cup can be thinner than other portions of the cup. In an example, a proximal outer ring can have a first elasticity level, a proximal inner ring can have a second elasticity level, and a distal concave portion can have a third elasticity level, wherein the second level is greater than the first level and the third level is greater than the second level. In an example, a distal concave portion can have a dorsal-to-ventral depth between 1 and 3 inches. In an example, a distal concave portion of a smart bra cup can span between 30% and 70% of the dorsal-to-ventral depth of the cup. In an example, a distal concave portion can have a hemispherical, hemi-ellipsoidal, half oblate-spheroidal, or paraboloidal shape. In an example, the elasticity and/or flexibility of an distal concave portion can allow a person's breast to extend in a ventral (e.g. frontal) direction when the base of the breast is compressed by expansion of expandable chambers.

In an example, light emitters can be on a first side of a smart bra cup and light receivers can be on a second side (e.g. the opposite side) of the cup, wherein light from the light emitters is transmitted through breast tissue before being received by the light receivers. In an example, light emitters can be on a lower side of a smart bra cup and light receivers can be on an upper side of the cup, wherein light from the light emitters is transmitted through breast tissue before being received by the light receivers. In an example, light emitters can be on a right side of a smart bra cup and light receivers can be on a left side of the cup, wherein light from the light emitters is transmitted through breast tissue before being received by the light receivers. In an example, light emitters can be on a first side of an oblique virtual plane through a smart bra cup and light receivers can be on a second side (e.g. the opposite side) of the oblique virtual plane, wherein light from the light emitters is transmitted through breast tissue before being received by the light receivers.

In an example, there can be rings of optical sensors around the inner circumference of a proximal inner ring, wherein each ring further comprises a plurality of light emitters and light receivers. In an example, there can be rings of optical sensors around the inner circumference of a proximal inner ring, wherein each ring further comprises an alternating sequence of light emitters and light receivers. In an example, there can be rings of optical sensors around the circumference of a proximal inner ring, wherein each ring further comprises a sequence of different-color light emitters and light receivers.

In an example, there can be light emitters and light receivers on both a proximal inner ring and on a distal concave portion of a bra cup. In another example, there can be light emitters and light receivers only on the proximal inner ring. In an example, the density of light emitters and/or light receivers can be greater in proximal portions of a smart bra cup than in distal portions of the cup. In an example, the density of light emitters and/or light receivers can be greater in selected quadrants (e.g. the upper-outer quadrant) of a smart bra cup than in other quadrants of the cup. In an example, different light emitters can be activated at different times to create different light transmission vectors between light emitters and light receivers at different times. In an example, light emitters at different radial locations on a smart bra cup can be activated at different times. In an example, different light emitters with different colors and/or spectral ranges can be activated at different times. In an example, the same light emitter can emit light with different colors and/or spectral ranges at different times.

In an example, a smart bra cup can comprise: a proximal outer ring which is part of a smart bra cup, wherein the outer ring is configured to encircle a person's breast; a proximal inner ring which is part of the smart bra cup, wherein the inner ring is configured to encircle the person's breast and wherein the inner ring is inside the outer ring; a plurality of expandable chambers between the inner ring and the outer ring; and a plurality of optical sensors (including light emitters and light receivers) between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

In an example, a smart bra cup can comprise: a proximal outer ring which is part of a smart bra cup, wherein the outer ring is configured to encircle a person's breast; two concave segments on opposite sides of the smart bra cup, wherein the concave segments are inside the outer ring; a plurality of expandable chambers between the concave segments and the outer ring; and a plurality of optical sensors (including light emitters and light receivers) between the concave segments and the person's breast, wherein changes in light emitted from the light emitters and received by the light receivers which are caused by transmission of the light through breast tissue are analyzed to detect, locate, evaluate, characterize, and/or image abnormal breast tissue. Relevant variations discussed elsewhere in this disclosure or in priority-linked disclosures can also be applied to this example.

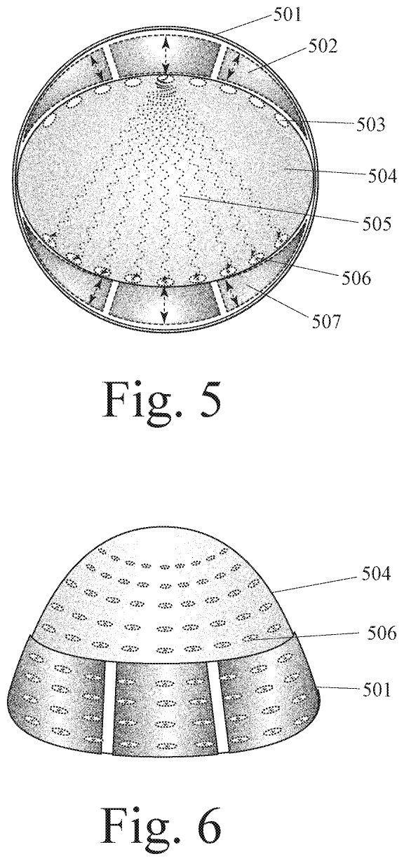

FIGS. 5 and 6 show two views, from two difference perspectives, of another example of a smart bra cup with a plurality of expandable chambers and optical sensors on either side of the cup for detecting abnormal breast tissue. FIG. 5 shows a semi-transparent frontal view of this smart bra cup. FIG. 6 shows an upward-facing view of this smart bra cup.

With respect to components, FIGS. 5 and 6 show two views of a smart bra cup comprising: (a) a proximal outer ring 501 which is part of a smart bra cup, wherein the outer ring is configured to encircle a person's breast; (b) a plurality of expandable chambers (including 502 and 507) on each side (e.g. on opposite sides) of the cup within the inner ring; (c) an elastic concave distal portion 504 of the smart bra cup which covers a distal portion of the person's breast; and (e) a plurality of light emitters 503 and light receivers 506 between the expandable chambers and the person's breast, wherein changes in light 505 emitted from the light emitters and received by the light receivers which are caused by transmission of the light through breast tissue are analyzed to detect, locate, evaluate, characterize, and/or image abnormal breast tissue.

More generally, a smart bra cup can comprise: a proximal outer ring which is part of a smart bra cup, wherein the outer ring is configured to encircle a person's breast; a plurality of expandable chambers on each side (e.g. on opposite sides) of the cup within the inner ring; an elastic concave distal portion of the smart bra cup which covers a distal portion of the person's breast; and a plurality of light emitters and light receivers between the expandable chambers and the person's breast, wherein changes in light emitted from the light emitters and received by the light receivers which are caused by transmission of the light through breast tissue are analyzed to detect, locate, evaluate, characterize, and/or image abnormal breast tissue.

In an example, both the right and left side cups of a smart bra can have optical sensors for detecting abnormal breast tissue. In an example, the bra cup design shown in FIGS. 5 and 6 can be used for one or both cups. In an example, optical sensors can be permanently integrated into a smart bra cup. In another example, a bra cup can have a pocket, pouch, or compartment into which optical sensors (and electronics) can be placed, wherein the optical sensors can be removed before the bra is washed and put back after the bra has been washed. In another example, this device can be embodied in a smart bra cup insert which is removably-inserted between a cup of a conventional bra and a person's breast.

In an example, a smart bra can further comprise a pump and tubes. The pump pumps a flowable substance (e.g. gas, liquid, or gel) through the tubes into expandable chambers. Expansion of the expandable chambers gently compresses a person's breast for more accurate optical scanning over shorter distances. In another example, a pump can be a separate component which is removably-connected to the smart bra. In an example, the person wearing the bra can actively control the operation of a pump so that it can be stopped (or reversed) if compression of the person's breast becomes uncomfortable. In an example, a smart bra can further comprise one or more additional components selected from the group consisting of: a pump, a power source (e.g. battery), electroconductive pathways which transmit electrical energy to the light emitters, a data processor, a data transmitter, a data receiver, a pressure sensor, a motion sensor, a piezoelectric actuators, and an electromagnetic actuator. In an example, one or more of these additional components can be located on a back strap of the smart bra.

In this example, proximal is defined as being closer to a person's chest wall and distal is defined as being farther from the person's chest wall. In an example, a proximal outer ring can encircle a person's breast adjacent to (and/or in contact with) the person's chest wall. In an example, a proximal outer ring can be relatively rigid (e.g. more rigid than the rest of the bra cup). In an example, a proximal outer ring can have a dorsal-to-ventral width between 1 and 3 inches. In an example, a proximal outer ring can span between 20% and 70% of the dorsal-to-ventral depth of a smart bra cup. In an example, a proximal outer ring can have a frustal, cylindrical, annular, or toroidal shape. In an example, the cross-sectional perimeter of a proximal outer ring can have a circular, oblate-circular, elliptical, oval, or teardrop shape.

In an example, a cross-sectional perimeter of a proximal outer ring can have a longitudinal axis. In an example, the longitudinal axis of a proximal outer ring can be parallel to a bustline vector which connects the apexes of right and left side bra cups. In another example, the longitudinal axis of a proximal outer ring can be perpendicular to a line which is parallel to the bustline vector. In another example, the longitudinal axis of a proximal outer ring can intersect a line which is parallel to the bustline vector at an angle between 30 and 60 degrees.

In an example, an expandable chamber can be an inflatable chamber (e.g. balloon). In an example, an expandable chamber can be expanded by being filled with a gas (e.g. air). In an example, an expandable chamber can be a fluid-filled chamber. In an example, an expandable chamber can be expanded by being filled with a liquid (e.g. saline solution). In an example, an expandable chamber can have a keystone, trapezoidal, or annular section shape. In this example, there can be multiple expandable chambers on each side of a smart bra cup.

In this example, there are two sets of three expandable chambers, wherein there is one set of three expandable chambers on each side (e.g. on opposite sides) of a smart bra cup. In this example, a middle expandable chamber between two other chambers in a set of chambers is larger than the other two chambers. In an example, a middle expandable chamber between two other chambers in a set of chambers can be expanded more than the other two chambers. In this example, a plurality of expandable chambers on a given side of a smart bra cup can collectively (e.g. together) comprise a crescent or banana shape.

In an example, a smart bra cup can further comprise a pressure sensor whose data is used to limit (or otherwise control) the expansion of an expandable chamber. In an example, a pressure sensor can be in fluid communication with the interior of an expandable chamber. In another example, a pressure sensor can measure the pressure between a smart bra cup and a person's breast. In an example, a smart bra can automatically shut off a pump when the pressure within an expandable chamber or between a smart bra cup and a person's breast exceeds a selected level.

In an example, a distal concave portion (e.g. distal dome) of a smart bra cup can be more elastic, more flexible, and/or less rigid than other portions of the cup. In an example, a distal concave portion (e.g. distal dome) of a smart bra cup can be thinner than other portions of the cup. In an example, a distal concave portion can have a dorsal-to-ventral depth between 1 and 3 inches. In an example, a distal concave portion of a smart bra cup can span between 30% and 70% of the dorsal-to-ventral depth of the cup. In an example, a distal concave portion can have a hemispherical, hemi-ellipsoidal, half oblate-spheroidal, or paraboloidal shape. In an example, the elasticity and/or flexibility of an distal concave portion can allow a person's breast to extend in a ventral (e.g. frontal) direction when the base of the breast is compressed by expansion of expandable chambers.

In an example, light emitters can be on a first side of a smart bra cup and light receivers can be on a second side (e.g. the opposite side) of the cup, wherein light from the light emitters is transmitted through breast tissue before being received by the light receivers. In an example, light emitters can be on a lower side of a smart bra cup and light receivers can be on an upper side of the cup, wherein light from the light emitters is transmitted through breast tissue before being received by the light receivers. In an example, light emitters can be on a right side of a smart bra cup and light receivers can be on a left side of the cup, wherein light from the light emitters is transmitted through breast tissue before being received by the light receivers. In an example, light emitters can be on a first side of an oblique virtual plane through a smart bra cup and light receivers can be on a second side (e.g. the opposite side) of the oblique virtual plane, wherein light from the light emitters is transmitted through breast tissue before being received by the light receivers.

In an example, there can be rings of optical sensors around the inner circumference of a cup, wherein each ring further comprises a plurality of light emitters and light receivers. In an example, there can be rings of optical sensors around the inner circumference of a cup, wherein each ring further comprises an alternating sequence of light emitters and light receivers. In an example, there can be rings of optical sensors around the circumference of a cup, wherein each ring further comprises a sequence of different-color light emitters and light receivers.

In an example, the density of light emitters and/or light receivers can be greater in proximal portions of a smart bra cup than in distal portions of the cup. In an example, the density of light emitters and/or light receivers can be greater in selected quadrants (e.g. the upper-outer quadrant) of a smart bra cup than in other quadrants of the cup. In an example, different light emitters can be activated at different times to create different light transmission vectors between light emitters and light receivers at different times. In an example, light emitters at different radial locations on a smart bra cup can be activated at different times. In an example, different light emitters with different colors and/or spectral ranges can be activated at different times. In an example, the same light emitter can emit light with different colors and/or spectral ranges at different times. Relevant variations discussed elsewhere in this disclosure or in priority-linked disclosures can also be applied to this example.

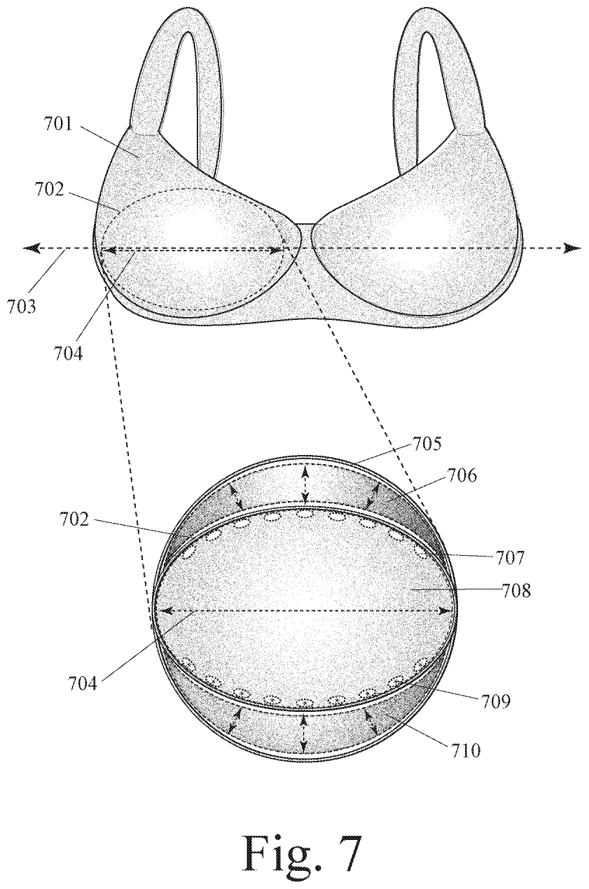

FIG. 7 shows two views of an example of a smart bra cup with expandable chambers and optical sensors on either side of horizontal plane (e.g. horizontal when worn by a person who is standing up) through the cup. The upper portion of FIG. 7 shows a frontal view of the entire bra 701, including bustline vector 703 which passes through the apexes of right-side and left-side bra cups. The lower portion of FIG. 7 shows a close-up semi-transparent frontal view of one of the bra cups, including views of proximal rings, expandable chambers, light emitters, and light receivers on the cup.

With respect to components, FIG. 7 shows a smart bra cup comprising: (a) a proximal outer ring 705 which is part of a cup on a smart bra 701, wherein the outer ring is configured to encircle a person's breast; (b) a proximal inner ring 702 which is part of the cup, wherein the inner ring is configured to encircle the person's breast, wherein the inner ring is inside the outer ring, wherein a bustline vector 703 of the smart bra is a line which passes through the apexes of the right-side and left-side bra cups of the bra, and wherein a longitudinal axis 704 of the inner ring is parallel to the bustline vector; (c) a plurality of expandable chambers (including 706 and 710) between the inner ring and the outer ring; (d) an elastic concave distal portion 708 of the cup which covers a distal portion of the person's breast; and (e) a plurality of light emitters 707 and light receivers 709 between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

More generally, a smart bra cup can comprise: a proximal outer ring which is part of a cup on a smart bra, wherein the outer ring is configured to encircle a person's breast; a proximal inner ring which is part of the cup, wherein the inner ring is configured to encircle the person's breast, wherein the inner ring is inside the outer ring, wherein a bustline vector of the smart bra is a line which passes through the apexes of the right-side and left-side bra cups of the bra, and wherein a longitudinal axis of the inner ring is parallel to the bustline vector; a plurality of expandable chambers (between the inner ring and the outer ring; (d) an elastic concave distal portion of the cup which covers a distal portion of the person's breast; and a plurality of light emitters and light receivers between the inner ring and the person's breast, wherein changes in light emitted from the light emitters and received by the light receivers which are caused by transmission of the light through breast tissue are analyzed to detect, locate, evaluate, characterize, and/or image abnormal breast tissue. Relevant variations discussed elsewhere in this disclosure or in priority-linked disclosures can also be applied to this example.

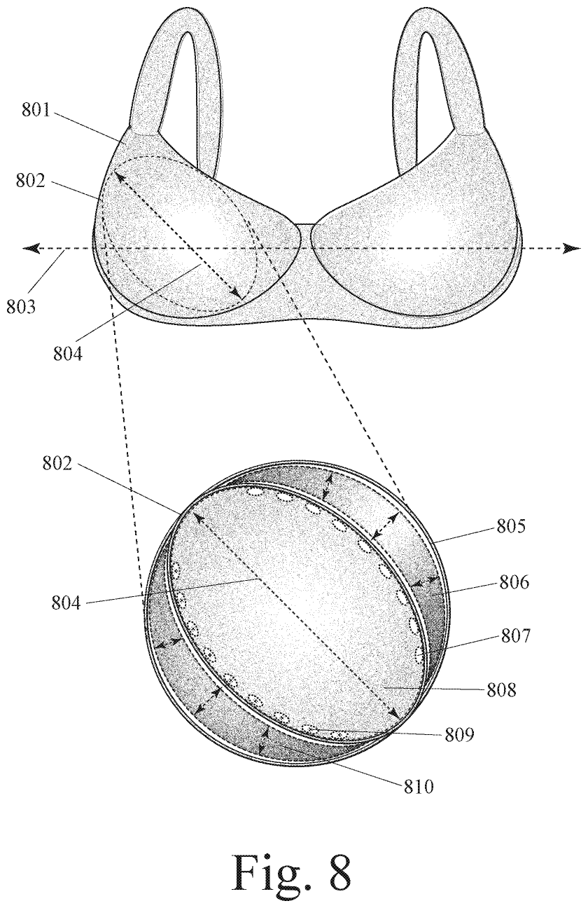

FIG. 8 shows two views of an example of a smart bra cup with expandable chambers and optical sensors on either side of oblique plane (e.g. between horizontal and vertical when worn by a person who is standing up) through the cup. The upper portion of FIG. 8 shows a frontal view of the entire bra 801, including bustline vector 803 which passes through the apexes of right-side and left-side bra cups. The lower portion of FIG. 8 shows a close-up semi-transparent frontal view of one of the bra cups, including views of proximal rings, expandable chambers, light emitters, and light receivers on the cup.

With respect to components, FIG. 8 shows a smart bra cup comprising: (a) a proximal outer ring 805 which is part of a cup on a smart bra 801, wherein the outer ring is configured to encircle a person's breast; (b) a proximal inner ring 802 which is part of the cup, wherein the inner ring is configured to encircle the person's breast, wherein the inner ring is inside the outer ring, wherein a bustline vector 803 of the smart bra is a line which passes through the apexes of the right-side and left-side bra cups of the bra, and wherein a longitudinal axis 804 of the inner ring intersects a line which is parallel to the bustline vector at an angle between 30 and 60 degrees; (c) a plurality of expandable chambers (including 806 and 810) between the inner ring and the outer ring; (d) an elastic concave distal portion 808 of the cup which covers a distal portion of the person's breast; and (e) a plurality of light emitters 807 and light receivers 809 between the inner ring and the person's breast, wherein changes in light emitted from the light emitters and received by the light receivers which are caused by transmission of the light through breast tissue are analyzed to detect, locate, evaluate, characterize, and/or image abnormal breast tissue. This design with expandable chambers and optical sensors (e.g. light emitters and detectors) on opposite sides of an oblique plane can be advantageous for better optical scanning and/or imaging of the upper-outer quadrant of the breast and the Tail of Spence, which are areas where abnormal tissue (e.g. tumors) are more likely to develop.

More generally, a smart bra cup can comprise: a proximal outer ring which is part of a cup on a smart bra, wherein the outer ring is configured to encircle a person's breast; a proximal inner ring which is part of the cup, wherein the inner ring is configured to encircle the person's breast, wherein the inner ring is inside the outer ring, wherein a bustline vector of the smart bra is a line which passes through the apexes of the right-side and left-side bra cups of the bra, and wherein a longitudinal axis of the inner ring is parallel to the bustline vector; a plurality of expandable chambers (between the inner ring and the outer ring; (d) an elastic concave distal portion of the cup which covers a distal portion of the person's breast; and a plurality of light emitters and light receivers between the inner ring and the person's breast, wherein changes in light emitted from the light emitters and received by the light receivers which are caused by transmission of the light through breast tissue are analyzed to detect, locate, evaluate, characterize, and/or image abnormal breast tissue. Relevant variations discussed elsewhere in this disclosure or in priority-linked disclosures can also be applied to this example.

I claim:

1. A smart bra cup comprising:
a proximal outer ring which is part of a smart bra cup, wherein the outer ring is configured to encircle a person's breast;
a plurality of expandable chambers inside the proximal outer ring; and
a plurality of light emitters and light receivers between the plurality of expandable chambers and the person's breast; wherein changes in light emitted from the light emitters and received by the light receivers which are caused by transmission of the light through breast tissue are analyzed to detect, locate, evaluate, characterize, and/or image abnormal breast tissue.

2. The smart bra cup in claim 1 wherein the proximal outer ring has a dorsal-to-ventral width between 1 and 3 inches.

3. The smart bra cup in claim 1 wherein the proximal outer ring spans between 20% and 70% of the dorsal-to-ventral depth of a smart bra cup.

4. The smart bra cup in claim 1 wherein the proximal outer ring has an annular shape.

5. The smart bra cup in claim 1 wherein the proximal outer ring has a frustal shape.

6. The smart bra cup in claim 1 wherein an expandable chamber has a crescent and/or banana shape.

7. The smart bra cup in claim 1 wherein the cup further comprises a proximal inner ring which is inside the proximal outer ring.

8. The smart bra cup in claim 7 wherein a longitudinal axis of the proximal inner ring is parallel to a bustline vector and wherein the bustline vector is a line which connects the apexes of right side and left side cups of a bra.

9. The smart bra cup in claim 7 wherein a longitudinal axis of the proximal inner ring intersects a line which is parallel to a bustline vector at an angle between 30 and 60 degrees and wherein the bustline vector is a line which connects the apexes of right side and left side cups of a bra.

10. The smart bra cup in claim 7 wherein there are multiple expandable chambers in each of two spaces between the proximal inner ring and the proximal outer ring.

11. A smart bra cup comprising:
a proximal outer ring which is part of a smart bra cup, wherein the outer ring is configured to encircle a person's breast;
a proximal inner ring which is part of the smart bra cup, wherein the inner ring is configured to encircle the person's breast and wherein the inner ring is inside the outer ring;
a plurality of expandable chambers between the inner ring and the outer ring;
an elastic concave distal portion of the smart bra cup which covers a distal portion of the person's breast; and
a plurality of light emitters and light receivers between the inner ring and the person's breast, wherein changes in light emitted from the light emitters and received by the light receivers which are caused by transmission of the light through breast tissue are analyzed to detect, locate, evaluate, characterize, and/or image abnormal breast tissue.

12. The smart bra cup in claim 11 wherein the proximal outer ring has a dorsal-to-ventral width between 1 and 3 inches.

13. The smart bra cup in claim 11 wherein the proximal outer ring spans between 20% and 70% of the dorsal-to-ventral depth of a smart bra cup.

14. The smart bra cup in claim 11 wherein the proximal outer ring has an annular shape.

15. The smart bra cup in claim 11 wherein the proximal outer ring has a frustal shape.

16. The smart bra cup in claim 11 wherein a longitudinal axis of the proximal inner ring is parallel to a bustline vector and wherein the bustline vector is a line which connects the apexes of right side and left side cups of a bra.

17. The smart bra cup in claim 11 wherein a longitudinal axis of the proximal inner ring intersects a line which is parallel to a bustline vector at an angle between 30 and 60 degrees and wherein the bustline vector is a line which connects the apexes of right side and left side cups of a bra.

18. The smart bra cup in claim 11 wherein an expandable chamber has a crescent and/or banana shape.

19. The smart bra cup in claim 11 wherein there are multiple expandable chambers in each of two spaces between the proximal inner ring and the proximal outer ring.

20. A smart cup insert comprising:
a cup insert which is configured to be removably inserted between the cup of a bra and a person's breast;
wherein the cup insert further comprises a plurality of expandable chambers; and
wherein the cup insert further comprises a plurality of light emitters and light receivers; and wherein changes in light emitted from the light emitters and received by the light receivers which are caused by transmission of the light through breast tissue are analyzed to detect, locate, evaluate, characterize, and/or image abnormal breast tissue.
